SEQUENCE LISTING (4 sequences)

SEQ ID NO: 1 - protein, 119 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 2 - protein, 110 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 3 - protein, 449 aa, synthetic construct
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 217 aa, synthetic construct
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0284215 A1
(43) Pub. Date: Sep. 24, 2026

(54) DOSING ANTI-FOLATE RECEPTOR ALPHA ANTIBODY DRUG CONJUGATES

(71) Applicant: Eli Lilly and Company, Indianapolis, IN (US)

(72) Inventors: Emin AVSAR, New York, NY (US); Claire Frances FRIEDMAN, New York, NY (US); John PAGEL, Seattle, WA (US)

(21) Appl. No.: 19/570,694

(22) Filed: Mar. 18, 2026

Related U.S. Application Data
(60) Provisional application No. 63/774,402, filed on Mar. 19, 2025.
(60) Provisional application No. 63/789,198, filed on Apr. 15, 2025.
(60) Provisional application No. 63/808,880, filed on May 20, 2025.
(60) Provisional application No. 63/860,023, filed on Aug. 8, 2025.
(60) Provisional application No. 63/887,654, filed on Sep. 25, 2025.
(60) Provisional application No. 63/917,477, filed on Nov. 14, 2025.
(60) Provisional application No. 63/960,835, filed on Jan. 15, 2026.

(51) Int. Cl. A61K 47/68; A61K 39/00; A61P 35/00; C07K 16/22; C07K 16/28

(52) U.S. Cl. CPC A61K 47/68037; A61K 47/6849; A61K 47/6889; A61P 35/00; C07K 16/22; C07K 16/28; A61K 2039/505

(57) ABSTRACT

The present invention relates to methods of treatment of subjects suffering from cancers that express folate receptor α using anti-folate receptor α antibody drug conjugates.

### CROSS REFERENCE

This application claims the benefit of U.S. Provisional Application No. 63/774,402 filed Mar. 19, 2025, U.S. Provisional Application No. 63/789,198 filed Apr. 15, 2025, U.S. Provisional Application No. 63/808,880 filed May 20, 2025, U.S. Provisional Application No. 63/860,023 filed Aug. 8, 2025, U.S. Provisional Application No. 63/887,654 filed Sep. 25, 2025, U.S. Provisional Application No. 63/917,477 filed Nov. 14, 2025, and U.S. Provisional Application No. 63/960,835 filed Jan. 15, 2026, all of which are incorporated by reference herein in their entireties.

### SEQUENCE LISTING

The instant application contains a Sequence Listing in XML format with a file name txt_31461_WO, created on Jan. 14, 2026 and with a file size of 2,601 bytes which is hereby incorporated by reference in its entirety.

### BACKGROUND

The present disclosure relates to methods of treating subjects suffering from cancers that express folate receptor α (FRα) using anti-folate receptor α antibody drug conjugates (anti-FRα ADCs). Folate receptor α (FRα) is generally displayed on the apical surface of polarized epithelial cells, particularly in the proximal tubule cells of the kidney, in the choroid plexus, and in glomerular endothelial cells, but absent in most other healthy tissues (Wu et al. Cancer Epidemiol Biomarkers Prev. 1999; 8 (9): 775; Weitman et al. Cancer Res. 1992; 52 (23): 6708). Non-malignant tissue has a limited distribution of FRα. However, various cancer types, including ovarian, endometrial, cervical, triple-negative breast, and lung cancers, are characterized by the overexpression of FRα, which makes this receptor α promising anticancer drug target (Yazaki et al. J Gynecol Oncol. 2022; 33 (6): e82; Scaranti et al. Nat Rev clin Concol. 2020; 17 (6): 349; Cheung et al. Oncotarget. 2016; 7 (32): 52553). Specifically, FRα expression as determined by IHC has been described in up to 70% to 90% of ovarian, 80% of NSCLC, 30% to 40% of breast (71% to 86% in TNBC), 20% to 50% in endometrial, 37% to 80% in pancreatic, and 30% to 40% of colorectal cancers. Expression of FRα has also been found in metastases from these diseases (see The Human Protein Atlas website for ENSG00000110195-FOLR1; Yazaki et al. 2022; Norton et al. NPJ Breast Cancer 2020; 6:4; Omote et al. Med Mol Morphol. 2018; 51 (4): 237; Cai et al. Oncotarget. 2017; 8 (23): 37646; Boogerd et al. Oncotarget. 2016; 7 (14): 17442; O'Shannessy et al. Oncotarget. 2012; 3 (4): 414), with higher levels of expression found in metastatic sites than in primary tumors in some histologies (Yazaki et al. 2022) and with expression levels correlating with survival (Norton et al. 2020; Cai et al. 2017).

Currently, mirvetuximab soravtansine-gynx (Elahere™) (MIRV) is the only anti-FRα ADC approved to treat FRα-positive, platinum-resistant epithelial ovarian, fallopian tube, or peritoneal cancer. MIRV consists of an FRα-binding antibody, a cleavable linker, and the cytotoxic maytansionoid effector molecule DM4 (Mai et al. Front Immunol. 2023; 14:1254532). In a Phase 3 trial comparing MIRV with chemotherapy in FRα positive platinum-resistant ovarian cancer (FORWARD I), MIRV did not meet its primary endpoint of progression-free survival. When patient subpopulations were analyzed, MIRV only benefitted those patients with a high FRα tumor expression, it was not active against low and negative FRα expressing cells (Matulonis U A., et al., J Clin Oncol 2023; 41:2436-45; Moore K N., et al., Ann Oncol 2021; 32:757-765). Based on the results in the subsequent SORAYA Phase 2/3 trial and MIRASOL Phase 3 trial, MIRV demonstrated encouraging activity as a monotherapy in platinum-resistant epithelial ovarian cancer patients whose tumors express high levels of FRα (Matulonis 2023; Moore 2023), however the toxicity profile demonstrated a high rate of ocular AEs, including blurred vision (40.8%) and keratopathy (32.1%) (Moore 2023).

There are other anti-FRα ADCs in clinical development, see for example, raludotatug deruxtecan (Daiichi Sankyo & Merck & Co.), rinatabart sesutecan (Rina-S, Genmab A/S), luveltamab tazevibulin (STRO-002 or Luvelta-Sutro Biopharma), farletuzuamb ecteribulin (MORAb-202-Eisai), AMT-151 (Multitude Therapeutics), PRO1184 (ProfoundBio), IMGN151 (Immunogen), AZD-5335 (Astrazeneca), IMGN-151 (Abbvie), ZW191 (Zymeworks), SYS604 (CSPC Megalith Biopharmaceutical), HMB9033 (Harbour Biomed), and BAT8006 (Bio-Thera Solutions).

Thus, there is a need to develop therapies to treat FRα positive cancers with an improved therapeutic index, to treat low-expressing and moderate-expressing in addition to high FRα expressing cancers, and a milder adverse event profile that will enable combination with earlier line cytotoxics and immune therapy.

### DESCRIPTION OF THE FIGURES

FIGS. 1A-D compares the in vivo activity of FRα ADC and MIRV, both at 3 mg/kg over days post treatment in various tumor models representing different FRα expression levels: high (FIG. 1A), moderate (FIGS. 1B and 1C) and low (FIG. 1D). See Example 1.

FIGS. 2A-B show the in vivo effect of FRα ADC in combination with bevacizumab at 1 mpk, 3 mpk or 5 mpk on efficacy in the OV-90 ovarian cancer model (FIG. 2A). Tumor volumes were measured every 3-4 days and plotted as average tumor volume (mm3)±standard error of mean (SEM) on the y-axis versus time for each treatment group (n=5). Body weight of each test animal in each test group was also monitored (FIG. 2B). Bev=bevacizumab; Mirv=mirvetuximab soravtansine-gynx. See Study A in Example 2.

FIGS. 3A-B show the in vivo effect of FRα ADC in combination with either cisplatin or cisplatin and bevacizumab at 1 mpk, 3 mpk or 5 mpk on efficacy in the OV-90 ovarian cancer model (FIG. 3A). Tumor volumes were measured every 3-4 days and plotted as average tumor volume (mm3)±standard error of mean (SEM) on the y-axis versus time for each treatment group (n=5). Body weight of each test animal in each test group was also monitored (FIG. 3B). Bev=bevacizumab; Mirv=mirvetuximab soravtansine-gynx. See Study B in Example 2.

FIG. 4 shows the Phase 1a trial scheme where CRC=colorectal cancer; DL=dose level; MIRV=mirvetuximab soravtansine-gynx; NSCLC=non-small cell lung cancer; and TNBC=triple-negative breast cancer. See Example 3.

FIG. 5 shows the Phase 1b trial scheme where CRC=colorectal cancer; NSCLC=non-small cell lung cancer; RP2D=recommended Phase 2 dose; and TNBC=triple-negative breast cancer. See Example 3.

FIG. 6 shows patient disease assessment results from the Phase Ia monotherapy dose escalation in high-grade serous ovarian cancer subjects (HGSOC) separated by FRα expression levels at 2 mg/kg, 3 mg/kg, 4 mg/kg and 6 mg/kg. “>” indicates ongoing treatment and “*” indicates patients who had prior MIRV treatment. See Example 3 and Tables 4 and 5.

FIG. 7 shows analyte concentrations in nM of free payload (exatecan), whole FRα ADC, and total antibody at different doses tested in the Phase Ia (2 mg/kg, 3 mg/kg, 4 mg/kg and 6 mg/kg) over time as measured in days. See Example 3 and Table 6.

FIG. 8 shows the Phase 3 study schema for platinum-resistant ovarian cancer (PROC). BRCA=breast cancer gene; IHC=immunohistochemistry; MIRV=mirvetuximab soravtansine. See Example 5.

FIG. 9 shows the Phase 3 study schema for platinum-sensitive ovarian cancer (PSOC). PLD=pegylated liposomal doxorubicin. See Example 5.

FIG. 10 shows the amended Phase Ia trial scheme discussed in Example 3 which adds Cohort A6 dose optimization for endometrial carcinoma adding substudies within Cohort A1: Arm A, Arm B and Arm C. See Example 6.

FIG. 11 shows the cycles in the study schema for newly added drug-drug interaction substudy Arm B in Cohort A1 in the amended Phase Ia trial scheme. See Example 7.

FIG. 12 shows the amended Phase Ib trial scheme discussed in Example 3 which modifies Cohort B dose expansion to add Cohorts B1, B2, B3 and B4. See Example 8.

### SUMMARY OF THE INVENTION

The present invention describes methods of treating subjects suffering from cancers that express folate receptor α (FRα) using anti-folate receptor α antibody drug conjugates.

Anchored to cellular membranes of non-malignant tissues, folate receptor alpha (FRα) is an internalizing target overexpressed in several solid tumors, including ovarian, endometrial, non-small cell lung cancer (NSCLC), triple-negative breast cancer (TNBC), pancreatic, and colorectal cancer (CRC). The FRα isoform has a high affinity for folates and folic acid, which, once bound to FRα, leads to internalization of clusters of ligand-receptor complexes within vesicles in the cell cytoplasm and release of folates. Interaction between folic acids and FRα has been linked to tumor progression. As FRα is known to have minimal physiological role after embryogenesis and in non-malignant tissues, its overexpression in tumor cells makes it a promising candidate to target in anticancer therapy. The differential expression of FRα on the surface of cancer cells within selected tumors, as opposed to non-malignant cells, enables the development of antibody-based therapeutic strategies including ADCs. These properties, together with the highly restricted expression of FRα on normal tissues and very efficient internalization rates, provides strong rationale for ADC-targeting of the FRα receptor in several FRα positive cancers.

ADCs are a selective drug delivery system that combines two classes of drugs with their complementing properties, with the goal of increasing the payload's therapeutic index and minimizing damage to normal tissues (Manzano and Ocaña 2020). Emerging evidence indicates that the efficacy of an ADC is dependent upon antibody-specific, linker-specific, and payload-specific factors, each of which is a function of complex interactions between the ADC and various components of the tumor and the tumor microenvironment.

MIRV is the only approved ADC that consists of an FRα-binding antibody, a cleavable linker, and the cytotoxic maytansinoid effector molecule DM4 (Mai et al. 2023). There are other anti-folate receptor alpha-antibody drug conjugates in clinical development, see for example, raludotatug deruxtecan (Daiichi Sankyo & Merck & Co.), rinatabart sesutecan (Rina-S, Genmab A/S), luveltamab tazevibulin (STRO-002 or Luvelta-Sutro Biopharma), farletuzuamb ecteribulin (MORAb-202-Eisai), AMT-151 (Multitude Therapeutics), PRO1184 (ProfoundBio), IMGN151 (Immunogen), AZD-5335 (Astrazeneca), IMGN-151 (Abbvie), ZW191 (Zymeworks), SYS604 (CSPC Megalith Biopharmaceutical), HMB9033 (Harbour Biomed), and BAT8006 (Bio-Thera Solutions).

WO2019/081455 describes the synthesis of the linker L of the present Formula III and is therefore incorporated by reference in its entirety. WO2023/170247 describes the synthesis of dipeptide-based drug linkers of the present Formula II as well as the conjugation of the resulting drug linkers to antibodies, such as the anti-FRα antibodies described herein to generate the anti-folate receptor alpha-antibody drug conjugates of the invention (Formula I) having a drug-antibody ratio (DAR) of 8 and thus is herein incorporated by reference in its entirety.

Ovarian cancer is a leading cause of cancer deaths among gynecologic malignancies worldwide, ranking third after cervical and uterine cancer. Debulking surgery and platinum-based chemotherapy are the initial standard of care treatment options. The most used platinum combinations are carboplatin plus paclitaxel or cisplatin plus paclitaxel, each of which have similar efficacy but different safety profiles. Cisplatin tends to have more renal, GI, and metabolic toxicity while carboplatin causes more thrombocytopenia and granulocytopenia (du Bois et al. J Natl Cancer Inst. 2003; 95 (17): 1320; Ozols et al. J Clin Oncol. 2003; 21 (17): 3194; Neijt et al. J Clin Oncol. 2000; 18 (17): 3084). Although the response rate to first-line platinum treatment is high, the majority of patients will experience disease recurrence (Wang et al. Signal Transduct Target Ther. 2020; 5 (1): 137).

Bevacizumab is a targeted cancer treatment developed against VEGF-A. Two randomized studies, GOG-0218 and ICON7, showed improvement of PFS when bevacizumab was added to carboplatin plus paclitaxel and continued as maintenance treatment until progression (Oza et al. Lancet Oncol. 2015; 16 (8): 928; Burger et al. N Engl J Med. 2011; 365:2473). However, in both studies no survival benefits were observed. Addition of bevacizumab to platinum doublet increases VEGFR-driven toxicity such as bleeding, hypertension, proteinuria, wound disruption, GI perforations, and thromboembolic events.

Endometrial cancer is one of only a few malignant conditions for which both incidence and mortality are currently rising. By 2040, it is projected to be the third most prevalent cancer and the fourth leading cause of cancer death among women (Siegel, R. L., et al., CA Cancer J clin 2023; 73:17-48). Standard first-line chemotherapy comprising paclitaxel plus carboplatin is used for the treatment of patients with advanced or recurrent endometrial cancer.

Pembrolizumab, also known as Keytruda®, is an immunotherapy drug that targets PD-1 to block the PD-1 receptor on T-cells. As a result of the Phase III KEYNOTE-868/NRG-GY018 clinical trial, pembrolizumab was approved for treating primary advanced or recurrent endometrial cancers as a single-agent therapy or in combination with carboplatin and paclitaxel (see Eskander, R. et al., N Engl J Med 388; 23 Jun. 8, 2023).

Itraconazole (ITZ) is a p-glycoprotein (P-gp) inhibitor commonly used as a cytochrome P450 3A (CYP3A4) inhibitor in drug-drug interaction (DDI) studies as CYP3A4 is an enzyme involved in the metabolism of many drugs, meaning it can increase the levels of other medications in a subject's body, potentially leading to increased side effects. It is approved as an antifungal medication used to treat and prevent fungal infections.

To overcome resistant or recurrent disease, and to mitigate observed toxicities with known treatments, the methods of the invention using the FRα ADC described herein are used to treat ovarian cancers and endometrial cancers, and other FRα positive cancers, both as monotherapy and in combination as described herein.

Embodiments of the methods of the present invention are contemplated to include, but are not limited to the following:

A method of treating a subject having a folate receptor alpha-positive cancer, the method comprising administering an anti-folate receptor alpha-antibody drug conjugate in a dosing regimen comprising: from about 1.0 mg/kg to about 10.0 mg/kg dose as an intravenous (IV) infusion on Day 1 of every 21-day cycle (Q3W) for at least 2 cycles, wherein the anti-folate receptor alpha-antibody drug conjugate has the chemical structure of Formula I: Ab-(L-D) p (I), wherein p is from 1-8. In another embodiment in the method above, the anti-folate receptor alpha-antibody drug conjugate has the chemical structure of Formula (II):

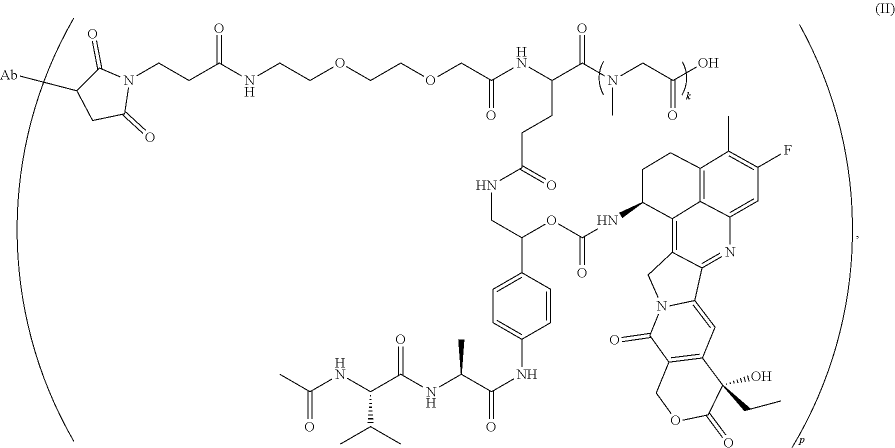

wherein Ab is an anti-folate receptor alpha antibody, k is from 2-20 and p is from 1-8.

In another embodiment, the method described above administers an anti-folate receptor alpha-antibody drug conjugate as described above in a dosing regimen from about 1.0 mg/kg to about 2.0 mg/kg, or from about 2.0 mg/kg to about 3.0 mg/kg, or from about 3.0 mg/kg to about 4.0 mg/kg, or from about 4.0 mg/kg to about 5.0 mg/kg, or from about 5.0 mg/kg to about 6.0 mg/kg, or from about 6.0 mg/kg to about 7.0 mg/kg, or about 7.0 mg/kg to about 8.0 mg/kg, or from about 8.0 mg/kg to about 9.0 mg/kg, or about 9.0 mg/kg to about 10.0 mg/kg, or from about 1 mg/kg to about 4 mg/kg, or from about 4 mg/kg to about 10 mg/kg as an intravenous (IV) infusion on Day 1 of every 21-day cycle (Q3W) for at least 2 cycles. In another embodiment, the method described above administers an anti-folate receptor alpha-antibody drug conjugate as described above in a dosing regimen from about 1.0 mg/kg to about 2.0 mg/kg, or from about 2.0 mg/kg to about 3.0 mg/kg, or from about 3.0 mg/kg to about 4.0 mg/kg, or from about 4.0 mg/kg to about 5.0 mg/kg, or from about 5.0 mg/kg to about 6.0 mg/kg, or from about 6.0 mg/kg to about 7.0 mg/kg, or about 7.0 mg/kg to about 8.0 mg/kg, or from about 8.0 mg/kg to about 9.0 mg/kg, or about 9.0 mg/kg to about 10.0 mg/kg, or from about 1 mg/kg to about 4 mg/kg, or from about 4 mg/kg to about 10 mg/kg as an intravenous (IV) infusion on Day 1 of every 28-day cycle (Q4W) for at least 2 cycles. In yet another embodiment, the method herein administers an anti-folate receptor alpha-antibody drug conjugate as described above in a dosing regimen comprising a dose of about 1.0 mg/kg, or about 1.5 mg/kg, or about 2.0 mg/kg, or about 2.5 mg/kg, or about 3.0 mg/kg, or about 3.5 mg/kg, or about 4.0 mg/kg, or about 4.5 mg/kg, or about 5.0 mg/kg, or about 5.5 mg/kg, or about 6.0 mg/kg, or about 7.0 mg/kg, or about 8.0 mg/kg, or about 9.0 mg/kg, or about 10.0 mg/kg, as an intravenous (IV) infusion on Day 1 of every 21-day cycle (Q3W) for at least 2 cycles. In still another embodiment, the method herein administers an anti-folate receptor alpha-antibody drug conjugate as described above in a dosing regimen comprising a dose of about 1.0 mg/kg, or about 1.5 mg/kg, or about 2.0 mg/kg, or about 2.5 mg/kg, or about 3.0 mg/kg, or about 3.5 mg/kg, or about 4.0 mg/kg, or about 4.5 mg/kg, or about 5.0 mg/kg, or about 5.5 mg/kg, or about 6.0 mg/kg, or about 7.0 mg/kg, or about 8.0 mg/kg, or about 9.0 mg/kg, or about 10.0 mg/kg, as an intravenous (IV) infusion on Day 1 of every 28-day cycle (Q4W) for at least 2 cycles. The doses disclosed herein are calculated based on actual body weight of the subject on Day 1 of treatment.

In another embodiment, with respect to the doses used as discussed above, such are administered as an intravenous (IV) infusion on Day 1 of every 21-day cycle (Q3W) for at least 2 cycles, or at least 3 cycles, or at least 4 cycles, or at least 5 cycles, or at least 6 cycles, or at least 7 cycles, or at least 8 cycles, or at least 9 cycles, or at least 10 cycles, or at least 11 cycles, or at least 12 cycles, or at least 13 cycles, or at least 14 cycles, or at least 15 cycles, or at least 16 cycles, or at least 17 cycles, or at least 18 cycles, or at least 19 cycles, or at least 20 cycles, or at least 21 cycles, or at least 22 cycles. In still another embodiment, with respect to the doses used as discussed above, such are administered as an intravenous (IV) infusion on Day 1 of every 28-day cycle (Q4W) for at least 2 cycles, or at least 3 cycles, or at least 4 cycles, or at least 5 cycles, or at least 6 cycles, or at least 7 cycles, or at least 8 cycles, or at least 9 cycles, or at least 10 cycles, or at least 11 cycles, or at least 12 cycles, or at least 13 cycles, or at least 14 cycles, or at least 15 cycles, or at least 16 cycles, or at least 17 cycles, or at least 18 cycles, or at least 19 cycles, or at least 20 cycles, or at least 21 cycles, or at least 22 cycles. The number of cycles may continue until disease progression or unacceptable toxicity as assessed by a healthcare provider.

In one embodiment, in any of the methods discussed above, the anti-FRα antibody Ab of Formula I may be, for example, the FRα antibodies disclosed in, for example, WO2005/080431, WO2008/031577, WO2009/132081, WO2011/106528, WO2012/033987, WO2014/087863, WO2018/071597, WO2019/177854, WO2019/177372, WO2020/016661, WO2020/127224, WO2022/223784, WO2022/109010, WO2023/178451, WO2023/169896, WO2023/066866, WO2023/116911, among others. In another embodiment, in the methods discussed above, the anti-FRα antibody Ab of Formula I discussed above may further comprise Fc silencing mutation(s). Such Fc silencing mutation(s) may include, for example, L234A and L235A according to the EU numbering scheme.

In another embodiment, in any of the methods discussed above, the anti-FRα antibody Ab of Formula I or Formula II comprise a variable heavy (VH) domain comprising the amino acid sequence of SEQ ID NO:1 and a variable light (VL) domain comprising the amino acid sequence of SEQ ID NO.2. In another embodiment, in the methods discussed above, the anti-FRα antibody Ab of Formula I or Formula II discussed herein may further comprise Fc silencing mutation(s). Such Fc silencing mutation(s) may include, for example, L234A and L235A according to the EU numbering scheme. In a further embodiment, in the methods discussed above, the anti-FRα antibody comprises a heavy chain (HC) comprising the amino acid sequence of SEQ ID NO:3 and a light chain (LC) comprising the amino acid sequence of SEQ ID NO: 4. In yet another embodiment, in the methods discussed herein where the anti-FRα antibody comprises a heavy chain (HC) comprising the amino acid sequence of SEQ ID NO:3 and a light chain (LC) comprising the amino acid sequence of SEQ ID NO:4, then k is 10 and p is 8 in Formula II.

In one embodiment, in any of the methods discussed above, D of Formula I is an inhibitor of topoisomerase I, including without limitation camptothecine analogues, indolocarbazole analogues, phenanthridine analogues, fluoroquinolone analogues, quinoxalines, evodiamines, acridines, naphthyridines, deoxynybomycin analogues, angustine analogues, stilbene thiazole analogues, pyrroloquinazolinoquinoline alkaloids or indenoisoquinoline analogues, Such topoisomerase I inhibitors may be selected from the group consisting of camptothecine and their analogues, including without limitation irinotecan, topotecan, camptothecine, SN-38, exatecan, DXd, silatecan, cositecan, lurtotecan, gimatecan, belotecan, or rubitecan.

In another embodiment, in any of the methods discussed above, L of Formula I is a polysarcosine of Formula (III):

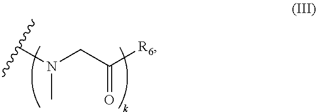

where k is an integer between 2 and 50, or between 4 and 30, or between 2 and 20, or between 2 and 10. In another embodiment, in Formula III k is 2, 3, 4, 5, 6, 7, 8, 9, 10, 11, 12, 13, 14, 15, 16, 17, 18, 19 or 20. In a further embodiment in Formula III, R₆ is OH or NH₂. In yet another embodiment, in the methods discussed above, L of Formula I or II is a polyethylene glycol moiety (PEG) comprising between 2 and 50 ethylene-glycol moieties. In another embodiment, L of Formula I is a cleavable linker with an optional stretcher unit linked to Ab and a cleavable moiety linked to D.

In one embodiment, in any of the methods discussed above, p of Formula I or Formula II is from 1 to 8, or from 1 to 4, or from 2 to 6, or from 2 to 8, or from 6 to 8. In another embodiment, in the methods discussed above, p of Formula I or Formula II is 1, 2, 3, 4, 5, 6, 7, 8, 9, or 10.

In one embodiment, in any of the methods discussed above, the FRα-positive cancer is characterized as a very low/ultra-low FRα expressing tumor, or a low FRα expressing tumor, or a moderate FRα expressing tumor, or a high FRα expressing tumor as measured by immunohistochemistry (IHC) as defined herein. Detection of FRα expression may be determined by, for example, immunohistochemistry or any of the FDA-approved tests for the measurement of FRα expression available which may include, without limitation, the Ventana FOLR1 (FOLR-2.1) RxDx Assay (Ventana Medical Systems, Inc.).

In one embodiment, in any of the methods discussed above, the FRα-positive cancer is selected from the group consisting of ovarian cancer, endometrial cancer, fallopian tube cancer, cervical cancer, non-small cell lung cancer (NSCLC), triple-negative breast cancer (TNBC), pancreatic cancer, primary peritoneal cancer, and colorectal cancer (CRC). In further embodiments, the ovarian cancer encompasses epithelial ovarian, fallopian tube or primary peritoneal cancers are platinum-resistant, where the subject has received at least one prior platinum-based treatment regimen. In additional embodiments, the epithelial ovarian fallopian tube or primary peritoneal cancers are platinum-sensitive, where the subject has received at least one prior platinum-based treatment regimen and a PARP inhibitor.

The FRα-positive ovarian cancers discussed above may be, for example but are not limited to: epithelial ovarian cancer, primary peritoneal cancer or fallopian tube cancer. The FRα-positive ovarian cancers discussed above may also be high grade serous ovarian cancer (HGSOC), low grade serous ovarian cancer (LGSOC), high-grade endometrioid ovarian carcinoma, ovarian carcinosarcoma, uterine serous carcinoma, mucinous ovarian carcinoma, epithelial ovarian cancer, borderline epithelial ovarian tumor, or ovarian clear-cell adenocarcinoma. Still other embodiments of FRα-positive ovarian cancers discussed above may also be malignant sex-cord stromal ovarian tumors or malignant germ cell ovarian tumors. In another embodiment, in any of the methods discussed above, the FRα-positive ovarian cancers discussed above may be platinum sensitive. In yet another embodiment, the FRα-positive ovarian cancers discussed above may be resistant to prior platinum treatment.

The FRα-positive endometrial cancers discussed above may be, for example but are not limited to: primary advanced endometrial cancer, recurrent endometrial carcinoma, endometrial stromal sarcoma, endometrial leiomyosarcoma, endometrial adenocarcinoma or endometrioid carcinoma. In one embodiment, the endometrial cancers to be treated are TP53-mutants. In another embodiment, the endometrial cancers to be treated are TP53-wild type. In other embodiments, the endometrial cancer may harbor deficient mismatch repair (dMMR) mutations, proficient mismatch repair (pMMR) mutations, microsatellite instability-high (MSI-H) mutations, or p53 mutations. In another embodiment, in any of the methods discussed above, the FRα-positive endometrial cancers discussed herein may be platinum sensitive. In yet another embodiment, the FRα-positive endometrial cancers discussed herein may be resistant to prior platinum treatment.

In one embodiment, in any of the methods discussed above, the FRα-positive cancer is metastatic. In another embodiment, in any of the methods discussed above, the FRα-positive cancer is recurrent.

In one embodiment, in any of the methods discussed above, the subject has failed to respond to at least one other therapy, prior to treatment with the anti-folate receptor alpha-antibody drug conjugate. In another embodiment, the subject was previously treated with MIRV or its biosimilar. In another embodiment if the subject is a platinum-resistant ovarian cancer (PROC) patient and was previously treated with MIRV or its biosimilar, the subject can be previously treated with ≤2 or ≤3 or ≤4 or ≤5 or ≤6 lines of therapy to receive the methods of the invention discussed herein. In another embodiment, the subject has not been treated previously with MIRV or its biosimilar (MIRV naive). In another embodiment, the subject has not been treated previously with a topoisomerase I inhibitor. In another embodiment, the subject was previously treated with ≤3 lines of therapy if the subject is a platinum-resistant ovarian cancer (PROC) patient. In yet another embodiment, the subject was previously treated with <3 lines of therapy in a platinum-resistant setting or the subject was previously treated with ≥3 lines of therapy in a platinum-resistant setting. In another embodiment, in any of the methods discussed above, if the subject is a platinum-sensitive ovarian cancer (PSOC) subject, the subject can be previously treated with ≥2 lines of therapy. In yet another embodiment, the subject was previously treated with a PARP inhibitor. In a further embodiment, the subject was not previously treated with a PARP inhibitor (PARPi naïve).

In one embodiment, in any of the methods discussed above, the subject is aged 65 and over.

In one embodiment, in any of the methods discussed above, the subject does not have platinum refractory disease, or any evidence of corneal keratopathy confirmed at baseline ocular assessment prior to the present treatment, or have a history of corneal transplant prior to the present treatment.

In one embodiment, in any of the methods discussed above, the anti-folate receptor alpha-antibody drug conjugate is administered as a monotherapy. In another embodiment, in any of the methods discussed above, the anti-folate receptor alpha-antibody drug conjugate monotherapy is administered to a subject with platinum-resistant ovarian cancer (PROC).

In one embodiment, for platinum-sensitive ovarian cancer subjects (PSOC), in any of the methods discussed above, the treatment further comprises administering an anti-VEGF antibody. In another embodiment, the anti-VEGF antibody is bevacizumab. In a further embodiment, the anti-VEGF antibody is a biosimilar to bevacizumab. In such a case, the bevacizumab or its biosimilar is administered after administration of the anti-folate receptor alpha-antibody drug conjugate and the dosing regimen for bevacizumab or its biosimilar is administered on Day 1 of every 21-day cycle (Q3W) at a dose of about 15 mg/kg for up to 22 cycles. In another embodiment, the combination with bevacizumab or its biosimilar is to treat metastatic disease as a first line therapy or to treat platinum sensitive disease as a second line therapy or to treat BRCAwt patients in a first line maintenance setting. In yet another embodiment, in any of the methods discussed above, the combination with bevacizumab or its biosimilar is used as first-line maintenance therapy or as second-line maintenance therapy in recurrent PSOC subjects.

In one embodiment, in any of the methods discussed above, the treatment further comprises administering an immune-oncology (IO) therapeutic in combination with the anti-folate receptor alpha-antibody drug conjugate. In another embodiment, the IO therapeutic is an anti-PD-1 antibody. In another embodiment, the anti-PD1 antibody is pembrolizumab or its biosimilar. If the method administers pembrolizumab or its biosimilar, then the pembrolizumab or its biosimilar is administered to the subject after administration of the anti-folate receptor alpha-antibody drug conjugate. Further if the method administers pembrolizumab or its biosimilar, the dosing regimen is administered on Day 1 of every 21-day cycle (Q3W) at a dose of about 200 mg or administered on Day 1 of every 6 weeks (Q6W) at a dose of about 400 mg. In yet another embodiment, the anti-PD1 antibody is dostarlimab or its biosimilar. In another embodiment, the anti-PD1 antibody is cemiplimab or its biosimilar. In a further embodiment, the anti-PD-1 antibody is nivolumab or its biosimilar. In yet another embodiment, the IO therapeutic is an anti-PD-L1 antibody. In another embodiment, the anti-PD-L1 antibody is atezolizumab or its biosimilar. In yet another embodiment, the anti-PD-L1 antibody is durvalumab or its biosimilar. In another embodiment, the anti-PD-L1 antibody is avelumab or its biosimilar. In a further embodiment, to the extent that the method comprises administering a combination of the anti-folate receptor alpha-antibody drug conjugate of the invention and pembrolizumab or its biosimilar, the subject being treated has a folate receptor alpha positive endometrial cancer. In another embodiment, the subject being treated has a folate receptor alpha positive endometrial cancer that is primary advanced or recurrent. In yet a further embodiment, the subject being treated has a folate receptor alpha positive endometrial cancer that is primary advanced endometrial cancer, recurrent endometrial carcinoma, endometrial stromal sarcoma, endometrial leiomyosarcoma, endometrial adenocarcinoma or endometrioid carcinoma. In one embodiment, the subject to be treated has an endometrial cancer harboring TP53-mutations. In another embodiment, the subject to be treated has an endometrial cancers that is TP53-wild type. In other embodiments, the subject to be treated has an endometrial cancer that harbors deficient mismatch repair (dMMR) mutations, proficient mismatch repair (pMMR) mutations, microsatellite instability-high (MSI-H) mutations, or p53 mutations. In another embodiment, in any of the methods discussed above, the subject to be treated has a platinum sensitive FRα-positive endometrial cancer. In yet another embodiment, the subject to be treated has a platinum resistant FRα-positive endometrial cancer.

In one embodiment, in any of the methods discussed above, the treatment further comprises administering a chemotherapeutic. In non-limiting examples, such chemotherapeutics may include paclitaxel, pegylated liposomal doxorubicin, gemcitabine or topotecan. In another embodiment, the chemotherapeutic is a platinum-containing compound. In yet another embodiment, the platinum-containing compound is carboplatin or cisplatin, where the carboplatin or cisplatin is administered after administration of the anti-folate receptor alpha-antibody drug conjugate where the dosing regimen for carboplatin or cisplatin is administered at AUC 4 on Day 1 of every 21-day cycle (Q3W). In a further embodiment, if the chemotherapeutic is carboplatin, then the anti-folate receptor alpha-antibody drug conjugate is administered on Day 1 of every 28-day cycle (Q4W), not Q3W. In another embodiment, the carboplatin or cisplatin is administered as described herein for up to 6 cycles. In a further embodiment, the carboplatin or cisplatin is administered after administration of the anti-folate receptor alpha-antibody drug conjugate. In another embodiment, the combination with carboplatin or cisplatin is to treat metastatic disease as a first line therapy. Alternatively, the combination with carboplatin or cisplatin is to treat platinum sensitive disease as a second line therapy. Other platinum-containing compounds include oxaliplatin or nedaplatin or dicycloplatin which may be used in the methods described herein.

In another embodiment, in any of the methods described above, the treatment further comprises administering a poly ADP-ribose polymerase (PARP) inhibitor. In non-limiting examples, such PARP inhibitors may include, for example olaparib, rucaparib, talazoparib, or niraparib. If the method contemplates a combination treatment with a PARP inhibitor then the PARP inhibitor is administered to the subject after administration of the anti-folate receptor alpha-antibody drug conjugate.

In one embodiment, an anti-folate receptor alpha-antibody drug conjugate as described in any of the methods discussed above for use in simultaneous, separate or sequential combination with an anti-VEGF antibody in the treatment of a FRα-positive cancer, as described above. In another embodiment, the anti-VEGF antibody is bevacizumab or its biosimilar, dosed separately after the anti-folate receptor alpha antibody drug conjugate is dosed. In another embodiment, bevacizumab or its biosimilar is dosed after the anti-folate receptor alpha antibody drug conjugate but within Day 1. In such a case, the dosing regimen for bevacizumab or its biosimilar is administered on Day 1 of every 21-day cycle (Q3W) at a dose of about 15 mg/kg for up to 22 cycles. In another embodiment, the combination with bevacizumab or its biosimilar is to (a) treat metastatic disease as a first line therapy or (b) to treat platinum sensitive disease as a second line therapy including treating those subjects with a known germline or somatic BRCA mutation who have had prior PARP inhibitor treatment or (c) to treat BRCAwt patients in a first line maintenance setting.

In one embodiment, an anti-folate receptor alpha-antibody drug conjugate as described in any of the methods discussed above for use in simultaneous, separate or sequential combination with a chemotherapeutic in the treatment of a FRα-positive cancer, as described above. In another embodiment, the chemotherapeutic is dosed separately after the anti-folate receptor alpha antibody drug conjugate is dosed. In non-limiting examples, such chemotherapeutics may include paclitaxel, pegylated liposomal doxorubicin, gemcitabine or topotecan. In another embodiment, the chemotherapeutic is a platinum-containing compound. In yet another embodiment, the platinum-containing compound is carboplatin or cisplatin, where the carboplatin or cisplatin is administered after administration of the anti-folate receptor alpha-antibody drug conjugate where the dosing regimen for carboplatin or cisplatin is administered on Day 1 of every 21-day cycle (Q3W) for up to 6 cycles. In another embodiment, the combination with carboplatin or cisplatin is to treat metastatic disease as a first line therapy. Alternatively, the combination with carboplatin or cisplatin is to treat platinum sensitive disease as a second line therapy. Other platinum-containing compounds include oxaliplatin or nedaplatin or dicycloplatin which may be used in the methods described herein.

In one embodiment the anti-folate receptor alpha-antibody drug conjugate as described in any of the methods discussed above is administered as a second-line treatment. In another embodiment, the anti-folate receptor alpha-antibody drug conjugate as described in any of the methods discussed above is administered as a third-line treatment. In another embodiment, the anti-folate receptor alpha-antibody drug conjugate as described in any of the methods discussed above is administered as a fourth-line treatment. In another embodiment, the anti-folate receptor alpha-antibody drug conjugate as described in any of the methods discussed above is administered as a fifth-line treatment. In another embodiment, the anti-folate receptor alpha-antibody drug conjugate as described in any of the methods discussed above is administered as a sixth-line treatment.

In one embodiment, in any of the methods discussed above, no pulmonary toxicity is observed in the subject as a result of treatment and/or no ocular toxicity is observed in the subject as a result of treatment and/or no neuropathy is observed in the subject as a result of treatment, and/or no alopecia is observed in the subject as a result of treatment. In a further embodiment, in any of the methods discussed above, no major drug-drug interactions are observed in the subject as a result of treatment.

In one embodiment, as a result of any of the methods discussed above, the ORR observed in the subject as a result of treatment is 30% or greater at 2 mg/kg, is 31% or greater at 2 mg/kg, is 32% or greater at 2 mg/kg, is 33% or greater at 2 mg/kg, is 35% or greater at 2 mg/kg, is 40% or greater at 2 mg/kg, is 45% or greater at 2 mg/kg, is 50% or greater at 2 mg/kg, is 25% or greater at 3 mg/kg, is 30% or greater at 3 mg/kg, is 35% or greater at 3 mg/kg, is 36% or greater at 3 mg/kg, is 37% or greater at 3 mg/kg, is 38% or greater at 3 mg/kg, is 39% or greater at 3 mg/kg, is 40% or greater at 3 mg/kg, is 45% or greater at 3 mg/kg, is 50% or greater at 3 mg/kg, is 55% or greater at 3 mg/kg, is 60% or greater at 3 mg/kg, is 30% or greater at 4 mg/kg, is 35% or greater at 4 mg/kg, is 40% or greater at 4 mg/kg, is 45% or greater at 4 mg/kg, is 50% or greater at 4 mg/kg, is 55% or greater at 4 mg/kg, is 60% or greater at 4 mg/kg, is 61% or greater at 4 mg/kg, is 50% or greater at 6 mg/kg, is 55% or greater at 6 mg/kg, is 56% or greater at 6 mg/kg.

In one embodiment, in any of the methods discussed above, when the FRα-positive cancer is characterized as a high FRα expressing tumor as measured by immunohistochemistry (IHC), the ORR observed in the subject as a result of treatment is 50% or greater at 2 mg/kg or at 3 mg/kg or at 4 mg/kg or at 6 mg/kg. In another embodiment, in any of the methods discussed above, when the FRα-positive cancer is characterized as a moderate FRα expressing tumor as measured by immunohistochemistry (IHC), the ORR observed in the subject as a result of treatment is 40% or greater at 2 mg/kg or at 3 mg/kg or at 4 mg/kg or at 6 mg/kg. In another embodiment, in any of the methods discussed above, when the FRα-positive cancer is characterized as a low FRα expressing tumor as measured by immunohistochemistry (IHC), the ORR observed in the subject as a result of treatment is 40% or greater at 2 mg/kg or at 3 mg/kg or at 4 mg/kg or at 6 mg/kg. In another embodiment, in any of the methods discussed above, when the FRα-positive cancer is characterized as an ultra low FRα expressing tumor as measured by immunohistochemistry (IHC), the ORR observed in the subject as a result of treatment is 40% or greater at 2 mg/kg or at 3 mg/kg or at 4 mg/kg or at 6 mg/kg.

In one embodiment, in any of the methods discussed above, the ORR observed in the subject as a result of treatment is 40% or greater at 2 mg/kg or at 3 mg/kg or at 4 mg/kg or at 6 mg/kg when the subject was previously treated with MIRV. In another embodiment, in any of the methods discussed above, the ORR observed in the subject as a result of treatment is 45% or greater at 2 mg/kg or at 3 mg/kg or at 4 mg/kg or at 6 mg/kg when the subject was previously treated with MIRV. In another embodiment, in any of the methods discussed above, the ORR observed in the subject as a result of treatment is 50% or greater at 2 mg/kg or at 3 mg/kg or at 4 mg/kg or at 6 mg/kg when the subject was previously treated with MIRV. In another embodiments, in any of the methods discussed above, the ORR observed in the subject as a result of treatment is 55% or greater at 2 mg/kg or at 3 mg/kg or at 4 mg/kg or at 6 mg/kg when the subject was previously treated with MIRV. In a further embodiment, in any of the methods discussed above, the ORR observed in the subject as a result of treatment is 60% or greater at 2 mg/kg or at 3 mg/kg or at 4 mg/kg or at 6 mg/kg when the subject was previously treated with MIRV.

In one embodiment, in any of the methods discussed above, the ORR observed in the subject as a result of treatment is 20% or greater at 2 mg/kg or at 3 mg/kg or at 4 mg/kg or at 6 mg/kg when the subject was not previously treated with MIRV (MIRV naïve). In another embodiment, in any of the methods discussed above, the ORR observed in the subject as a result of treatment is 30% or greater at 2 mg/kg or at 3 mg/kg or at 4 mg/kg or at 6 mg/kg when the subject was not previously treated with MIRV (MIRV naïve). In one embodiment, in any of the methods discussed above, the ORR observed in the subject as a result of treatment is 35% or greater at 2 mg/kg or at 3 mg/kg or at 4 mg/kg or at 6 mg/kg when the subject was not previously treated with MIRV (MIRV naïve). In one embodiment, in any of the methods discussed above, the ORR observed in the subject as a result of treatment is 40% or greater at 2 mg/kg or at 3 mg/kg or at 4 mg/kg or at 6 mg/kg when the subject was not previously treated with MIRV (MIRV naïve). In one embodiment, in any of the methods discussed above, the ORR observed in the subject as a result of treatment is 45% or greater at 2 mg/kg or at 3 mg/kg or at 4 mg/kg or at 6 mg/kg when the subject was not previously treated with MIRV (MIRV naïve).

In one embodiment, as a result of any of the methods discussed above, the disease control rate (DCR) observed in the subject as a result of treatment is 80% or greater at 2 mg/kg, is 81% or greater at 2 mg/kg, is 82% or greater at 2 mg/kg, is 83% or greater at 2 mg/kg, is 84% or greater at 2 mg/kg, is 85% or greater at 2 mg/kg, is 80% or greater at 3 mg/kg, is 81% or greater at 3 mg/kg, is 82% or greater at 3 mg/kg, is 80% or greater at 4 mg/kg, is 81% or greater at 4 mg/kg, is 82% or greater at 4 mg/kg, is 83% or greater at 4 mg/kg, is 85% or greater at 4 mg/kg, is 70% or greater at 6 mg/kg, is 71% or greater at 6 mg/kg, is 72% or greater at 6 mg/kg, is 73% or greater at 6 mg/kg, is 74% or greater at 6 mg/kg, is 75% or greater at 6 mg/kg.

In one embodiment, as a result of any of the methods discussed above, for platinum-resistant ovarian cancers, the mPFS efficacy is about between 5 months to about 6 months with an HR of 0.75 or lower, or about between 5 months to about 7 months with an HR of 0.65 or lower, or about 5 months to about 7 months with an HR of 0.70 or lower, or about between 5 months to about 6 months with an HR of 0.65 or lower. In another embodiment, as a result of any of the methods discussed above, for platinum-resistant ovarian cancers, the mOS efficacy is about between 14 months to about 15 months with an HR of 0.80 or lower, or about between 16 months to about 17 months with an HR of 0.70 or lower, or about between 17 months to about 18 months with an HR of 0.70 or lower.

In one embodiment, as a result of any of the methods discussed above, for platinum-sensitive ovarian cancers, the mPFS efficacy is about between 6 months to about 7 months with an HR of 0.70 or lower, or about between 7 months to about 8 months with an HR of 0.70 or lower, or about between 8 months to about 9 months with an HR of 0.70 or lower, or about between 9 months to about 10 months with an HR of 0.70 or lower, or about between 10 months to about 11 months with an HR of 0.70 or lower, or about between 11 months to about 12 months with an HR of 0.70 or lower, or about between 11 months to about 12 months with an HR of 0.70 or lower, about between 12 months to about 13 months with an HR of 0.75 or lower, or about between 12 months to about 13 months with an HR of 0.70 or lower, or about between 13 months to about 14 months with an HR of 0.75 or lower, or about between 13 months to about 14 months with an HR of 0.65 or lower, or about between 15 months to about 16 months with an HR of 0.65 or lower. In another embodiment, as a result of any of the methods discussed above, for platinum-sensitive ovarian cancers, the mOS efficacy is about between 37 months to about 38 months with an HR of 0.80 or lower, or about between 42 months to about 43 months with an HR of 0.70 or lower.

One embodiment is an anti-folate receptor alpha-antibody drug conjugate as described above for use in treating a folate receptor alpha-positive cancer in a subject as described above. Another embodiment is an anti-folate receptor alpha-antibody drug conjugate as described above for use in treating a folate receptor alpha-positive cancer in a subject as described above, wherein the anti-folate receptor alpha-antibody drug conjugate is administered in a dosing regimen comprising a dose from about 1.0 mg/kg to about 10.0 mg/kg as an intravenous (IV) infusion on Day 1 of every 21-day cycle (Q3W) for at least 2 cycles. Another embodiment is an anti-folate receptor alpha-antibody drug conjugate as described above for use in treating a folate receptor alpha-positive cancer in a subject as described above, wherein the anti-folate receptor alpha-antibody drug conjugate is administered in a dosing regimen comprising a dose from about 1.0 mg/kg to about 10.0 mg/kg as an intravenous (IV) infusion on Day 1 of every 28-day cycle (Q4W) for at least 2 cycles. Another embodiment is an anti-folate receptor alpha-antibody drug conjugate as described above for use in a method of treating a folate receptor alpha-positive cancer in a subject as described above, wherein the method comprises administering the anti-folate receptor alpha-antibody drug conjugate in a dosing regimen comprising a dose from about 1.0 mg/kg to about 10.0 mg/kg as an intravenous (IV) infusion on Day 1 of every 21-day cycle (Q3W) for at least 2 cycles. Another embodiment is an anti-folate receptor alpha-antibody drug conjugate as described above for use in a method of treating a folate receptor alpha-positive cancer in a subject as described above, wherein the method comprises administering the anti-folate receptor alpha-antibody drug conjugate in a dosing regimen comprising a dose from about 1.0 mg/kg to about 10.0 mg/kg as an intravenous (IV) infusion on Day 1 of every 28-day cycle (Q4W) for at least 2 cycles. Another embodiment is use of an anti-folate receptor alpha-antibody drug conjugate as described above in the manufacture of a medicament for treating a folate receptor alpha-positive cancer in a subject as described above. Another embodiment is use of an anti-folate receptor alpha-antibody drug conjugate as described above in the manufacture of a medicament for treating a folate receptor alpha-positive cancer in a subject as described above, wherein the medicament is formulated for administration in a dosing regimen comprising a dose from about 1.0 mg/kg to about 10.0 mg/kg as an intravenous (IV) infusion on Day 1 of every 21-day cycle (Q3W) for at least 2 cycles. Another embodiment is use of an anti-folate receptor alpha-antibody drug conjugate as described above in the manufacture of a medicament for treating a folate receptor alpha-positive cancer in a subject as described above, wherein the medicament is formulated for administration in a dosing regimen comprising a dose from about 1.0 mg/kg to about 10.0 mg/kg as an intravenous (IV) infusion on Day 1 of every 28-day cycle (Q4W) for at least 2 cycles. Another embodiment is use of an anti-folate receptor alpha-antibody drug conjugate as described above for the manufacture of a medicament for treating a folate receptor alpha-positive cancer in a subject as described above, wherein the anti-folate receptor alpha-antibody drug conjugate is administered in a dosing regimen comprising a dose from about 1.0 mg/kg to about 10.0 mg/kg as an intravenous (IV) infusion on Day 1 of every 21-day cycle (Q3W) for at least 2 cycles. Another embodiment is use of an anti-folate receptor alpha-antibody drug conjugate as described above for the manufacture of a medicament for treating a folate receptor alpha-positive cancer in a subject as described above, wherein the anti-folate receptor alpha-antibody drug conjugate is administered in a dosing regimen comprising a dose from about 1.0 mg/kg to about 10.0 mg/kg as an intravenous (IV) infusion on Day 1 of every 28-day cycle (Q4W) for at least 2 cycles.

Additional contemplated embodiments of the present invention also include the following:

Embodiment 1: A method of treating a subject having a folate receptor alpha-positive cancer, the method comprising administering an anti-folate receptor alpha-antibody drug conjugate in a dosing regimen comprising: a dose from about 1.0 mg/kg to about 10.0 mg/kg dose as an intravenous (IV) infusion on Day 1 of every 21-day cycle (Q3W) for at least 2 cycles, wherein the anti-folate receptor alpha-antibody drug conjugate has the chemical structure of Formula II:

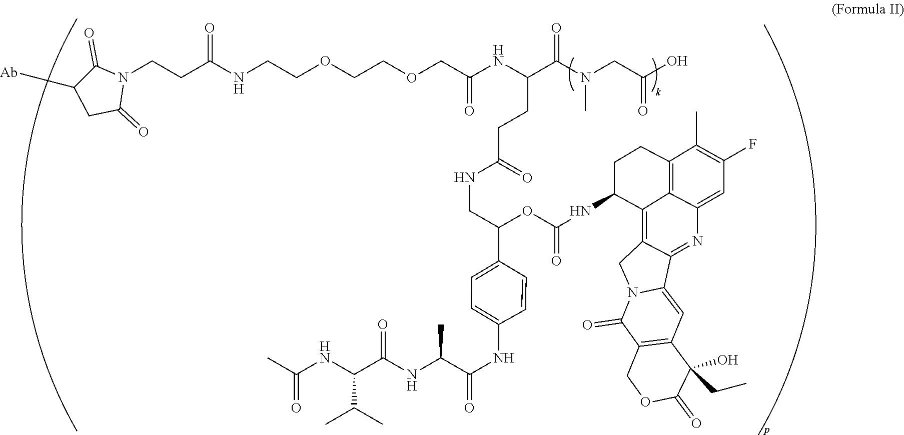

wherein Ab is an anti-FRα antibody, k is from 2-20 and p is from 1-8.

Embodiment 2: The method of embodiment 1, wherein the anti-FRα antibody comprises a variable heavy (VH) domain comprising the amino acid sequence of SEQ ID NO:1 and a variable light (VL) domain comprising the amino acid sequence of SEQ ID NO.2.

Embodiment 3: The method of embodiment 1, wherein the anti-FRα antibody further comprises Fc silencing mutation(s).

Embodiment 4: The method of embodiment 3, wherein the Fc silencing mutations are L234A and L235A.

Embodiment 5: The method of embodiment 1 or 2, wherein the anti-FRα antibody comprises a heavy chain (HC) comprising the amino acid sequence of SEQ ID NO:3 and a light chain (LC) comprising the amino acid sequence of SEQ ID NO:4.

Embodiment 6: The method of any one of embodiments 2-5, wherein k is 10 and p is 8.

Embodiment 7: The method of any one of embodiments 1-6, wherein the dose is from about 3.0 mg/kg to about 4.0 mg/kg dose.

Embodiment 8: The method of embodiment 7, wherein the dose is about 3.0 mg/kg.

Embodiment 9: The method of any one of embodiments 1-8, wherein the FRα-positive cancer is characterized as a low FRα expressing tumor, as measured by immunohistochemistry (IHC).

Embodiment 10: The method of any one of embodiments 1-8, wherein the FRα-positive cancer is characterized as a moderate FRα expressing tumor, as measured by immunohistochemistry (IHC).

Embodiment 11: The method of any one of embodiments 1-8, wherein the FRα-positive cancer is characterized as a high FRα expressing tumor, as measured by immunohistochemistry (IHC).

Embodiment 12: The method of any one of embodiments 1-11, wherein the FRα-positive cancer is selected from the group consisting of ovarian cancer, endometrial cancer, cervical cancer, non-small cell lung cancer (NSCLC), triple-negative breast cancer (TNBC), pancreatic cancer, and colorectal cancer (CRC).

Embodiment 13: The method of embodiment 12, wherein the ovarian cancer is epithelial ovarian cancer, primary peritoneal cancer, fallopian tube cancer, and high grade endometriod ovarian cancer, low grade serous ovarian cancer (LGSOC), and high grade serous ovarian cancer (HGSOC).

Embodiment 14: The method of embodiment 12 or 13, wherein the ovarian cancer is resistant to prior platinum treatment (PROC).

Embodiment 15: The method of embodiment 12 or 13, wherein the ovarian cancer is platinum sensitive (PSOC).

Embodiment 16: The method of any one of embodiments 1-15, wherein the subject was previously treated with mirvetuximab soravtansine-gynx or its biosimilar.

Embodiment 17: The method of any one of embodiments 1-14 or 16 wherein the subject was previously treated with ≤3 lines of therapy.

Embodiment 18: The method of embodiment 16 wherein the subject was previously treated with ≤4 lines of therapy.

Embodiment 19: The method of any one of embodiments 1-13 and 15-16, wherein the subject was previously treated with ≥2 lines of therapy.

Embodiment 20: The method of embodiment 19, wherein the subject was not previously treated with a poly ADP-ribose polymerase (PARP) inhibitor.

Embodiment 21: The method of any one of embodiments 1-20, wherein the subject has failed to respond to at least one other therapy, prior to treatment with the anti-folate receptor alpha-antibody drug conjugate.

Embodiment 22: The method of any one of embodiments 1-21, wherein the subject is aged 65 and over.

Embodiment 23: The method of any one of embodiments 1-22, wherein the FRα-positive cancer is metastatic.

Embodiment 24: The method of any one of embodiments 1-12 or 14-19, wherein the subject does not have platinum refractory disease.

Embodiment 25: The method of any one of embodiments 1-24, wherein the subject does not have evidence of corneal keratopathy confirmed at baseline ocular assessment.

Embodiment 26: The method of any one of embodiments 1-25, wherein the subject does not have a history of corneal transplant.

Embodiment 27: The method of any one of embodiments 1-14, 16-18, and 22-26, wherein the anti-folate receptor alpha-antibody drug conjugate is administered as a monotherapy.

Embodiment 28: The method of any one of embodiments 1-14, 16-18, and 22-26, wherein the treatment further comprises administering an anti-VEGF antibody.

Embodiment 29: The method of embodiment 28, wherein the anti-VEGF antibody is bevacizumab or its biosimilar.

Embodiment 30: The method of embodiment 29, wherein the bevacizumab or its biosimilar is administered after administration of the anti-folate receptor alpha-antibody drug conjugate.

Embodiment 31: The method of embodiment 29 or 30, wherein the dosing regimen for bevacizumab or its biosimilar is administered on Day 1 of every 21-day cycle (Q3W) at a dose of about 15 mg/kg for up to 22 cycles.

Embodiment 32: The method of any one of embodiments 29-31, wherein the combination with bevacizumab or its biosimilar is to treat metastatic disease as a first line therapy.

Embodiment 33: The method of any one of embodiments 29-31, wherein the combination with bevacizumab or its biosimilar is to treat platinum sensitive disease as a second line therapy.

Embodiment 34: The method of any one of embodiments 29-32, wherein the combination with bevacizumab or its biosimilar is the treat BRCAwt patients in a first line maintenance setting.

Embodiment 35: The method of any one of embodiments 1-14, 16-18, and 22-26, wherein the treatment further comprises administering an anti-PD-1 antibody or an anti-PD-L1 antibody.

Embodiment 36: The method of embodiment 35, wherein the anti-PD-1 antibody is pembrolizumab or its biosimilar.

Embodiment 37: The method of embodiment 36, wherein the pembrolizumab or its biosimilar is administered after administration of the anti-folate receptor alpha-antibody drug conjugate.

Embodiment 38: The method of embodiment 36 or 37, wherein the dosing regimen for pembrolizumab or its biosimilar is administered on Day 1 of every 21-day cycle (Q3W) at a dose of about 200 mg or administered on Day 1 of every 6 weeks (Q6W) at a dose of about 400 mg.

Embodiment 39: The method of any one of embodiments 35-38, wherein the FRα-positive cancer is endometrial cancer.

Embodiment 40: The method of embodiment 39, wherein the endometrial cancer is primary advanced or recurrent.

Embodiment 41: The method of any one of embodiments 1-14, 16-18, and 22-26, wherein the treatment further comprises administering carboplatin and the anti-folate receptor alpha-antibody drug conjugate is dosed on Day 1 of every 28-day cycle (Q4W).

Embodiment 42: The method of embodiment 41, wherein the carboplatin is dosed at AUC 4 on Day 1 of every 21-day cycle (Q3W).

Embodiment 43: The method of embodiment 42, wherein the carboplatin is administered after administration of the anti-folate receptor alpha-antibody drug conjugate.

Embodiment 44: The method of embodiment 43, wherein the subject is platinum-sensitive with ovarian cancer.

Embodiment 45: The method of any one of embodiments 1-44, wherein no pulmonary toxicity is observed in the subject as a result of treatment.

Embodiment 46: The method of any one of embodiments 1-45, wherein no neuropathy is observed in the subject as a result of treatment.

Embodiment 47: The method of any one of embodiments 1-46, wherein no ocular toxicity is observed in the subject as a result of treatment.

Embodiment 48: The method of any one of embodiments 1-47, wherein no major drug-drug interactions are observed in the subject as a result of treatment.

Embodiment 49: A method of treating a subject having a folate receptor alpha-positive cancer comprising administering an anti-folate receptor alpha-antibody drug conjugate in a dosing regimen comprising: a dose of 3.0 mg/kg as an intravenous (IV) infusion on Day 1 of every 21-day cycle (Q3W) for at least 2 cycles, wherein the anti-folate receptor alpha-antibody drug conjugate has the chemical structure of Formula II:

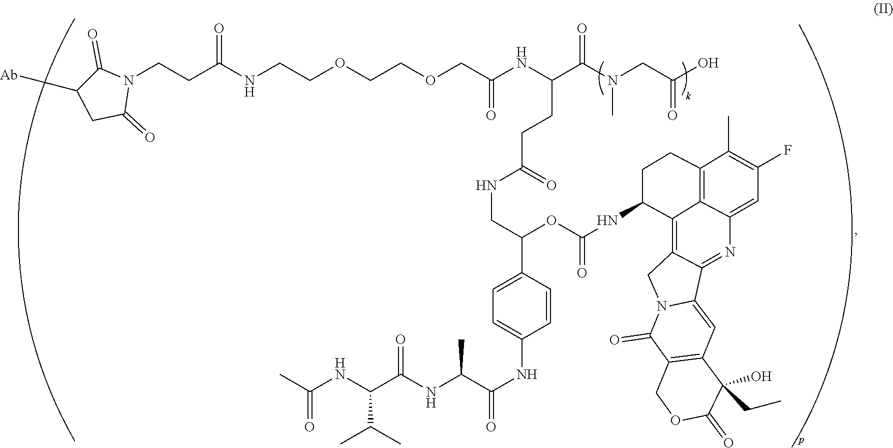

and
wherein Ab is an anti-FRα antibody comprises a heavy chain (HC) comprising the amino acid sequence of SEQ ID NO:3 and a light chain (LC) comprising the amino acid sequence of SEQ ID NO:4, k is 10 and p is 8, wherein the FRα-positive cancer is an ovarian cancer that is platinum resistant (PROC), wherein the subject received ≤3 prior lines of systemic cytotoxic therapy or ≤4 lines if MIRV was one of the prior lines of therapy.

Embodiment 50: A method of treating a subject having a folate receptor alpha-positive cancer comprising administering an anti-folate receptor alpha-antibody drug conjugate in a dosing regimen comprising: a dose of 3.0 mg/kg as an intravenous (IV) infusion on Day 1 of every 21-day cycle (Q3W) for at least 2 cycles, in combination with bevacizumab or its biosimilar which is administered after administration of the anti-folate receptor alpha-antibody drug conjugate, wherein the anti-folate receptor alpha-antibody drug conjugate has the chemical structure of Formula II:

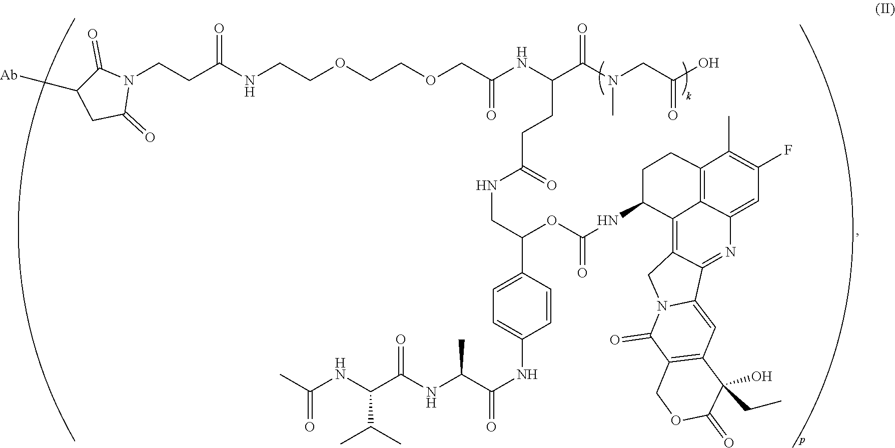

and
wherein Ab is an anti-FRα antibody comprises a heavy chain (HC) comprising the amino acid sequence of SEQ ID NO:3 and a light chain (LC) comprising the amino acid sequence of SEQ ID NO:4, k is 10 and p is 8, wherein the FRα-positive cancer is an ovarian cancer that is platinum sensitive (PSOC) wherein the subject received ≤2 prior lines of systemic cytotoxic therapy and had disease progression either during or within six months of completing treatment with a poly (ADP-ribose) polymerase inhibitor (PARPi).

### SEQUENCES

In one embodiment, the anti-FRα antibody amino acid sequences that comprise the FRα ADC of the invention are as follows:

TABLE 1
Anti-FRα Antibody Amino Acid Sequences for FRa ADC

| Description | Amino Acid Sequence | SEQ ID NO: |
|---|---|---|
| Variable Heavy (VH) | EVQLVESGGGVVQPGRSLRLSCSASGFTFSGYGLSWV | 1 |
| | RQAPGKGLEWVAMISSGGSYTYYADSVKGRFAISRDN | |
| | AKNTLFLQMDSLRPEDTGVYFCARHGDDPAWFAYWG | |
| | QGTPVTVSS | |
| Variable Light (VL) | DIQLTQSPSSLSASVGDRVTITCSVSSSISSNNLHWYQQ | 2 |
| | KPGKAPKPWIYGTSNLASGVPSRFSGSGSGTDYTFTISS | |
| | LQPEDIATYYCQQWSSYPYMYTFGQGTKVEIK | |
| Heavy Chain (HC) | EVQLVESGGGVVQPGRSLRLSCSASGFTFSGYGLSWV | 3 |
| | RQAPGKGLEWVAMISSGGSYTYYADSVKGRFAISRDN | |
| | AKNTLFLQMDSLRPEDTGVYFCARHGDDPAWFAYWG | |
| | QGTPVTVSSASTKGPSVFPLAPSSKSTSGGTAALGCLV | |
| | KDYFPEPVTVSWNSGALTSGVHTFPAVLQSSGLYSLSS | |
| | VVTVPSSSLGTQTYICNVNHKPSNTKVDKKVEPKSCD | |
| | KTHTCPPCPAPEAAGGPSVFLFPPKPKDTLMISRTPEVT | |
| | CVVVDVSHEDPEVKFNWYVDGVEVHNAKTKPREEQY | |
| | NSTYRVVSVLTVLHQDWLNGKEYKCKVSNKALPAPIE | |
| | KTISKAKGQPREPQVYTLPPSRDELTKNQVSLTCLVKG | |
| | FYPSDIAVEWESNGQPENNYKTTPPVLDSDGSFFLYSK | |
| | LTVDKSRWQQGNVFSCSVMHEALHNHYTQKSLSLSPG | |
| | K | |
| Light Chain (LC) | DIQLTQSPSSLSASVGDRVTITCSVSSSISSNNLHWYQQ | 4 |
| | KPGKAPKPWIYGTSNLASGVPSRFSGSGSGTDYTFTISS | |
| | LQPEDIATYYCQQWSSYPYMYTFGQGTKVEIKRTVAA | |
| | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |

### Definitions

The term “adverse event” or “AE” as used herein is any unfavorable medical occurrence in a participant administered an investigational product, which does not necessarily have a causal relationship with the treatment. An AE can therefore be any unfavorable and unintended sign, symptom, or disease temporally associated with the use of the investigational product, whether or not considered related to the investigational product. The severity of each AE may be graded using the NCI CTCAE v5.0. In the event of an AE for which no grading scale exists, the AE may be classified as mild (Grade 1), moderate (Grade 2), severe (Grade 3), life-threatening/debilitating (Grade 4), or fatal (Grade 5).

The term “antibody,” as used herein, refers to an isolated immunoglobulin molecule that specifically binds an antigen, such as, for example, a tumor antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4), unless otherwise specified.

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to schemes well known to those of skill in the art, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

The term “antibody drug conjugate” (ADC) as used herein refers to a class of compounds comprising (a) an antibody or antibody fragment, (2) a payload, such as a cytotoxic agent, an immunological modulator or therapeutic peptide, and (3) a linker that conjugates the antibody to the payload.

The term “ASCO/CAP HER2 testing guidelines” as used herein refers to HER2 testing guidelines in breast cancer to ensure that patients receive appropriate HER2-targeted therapies. See Wolff AC, Somerfield M R, Dowsett M, et al. Human epidermal growth factor receptor 2 testing in breast cancer: ASCO-CAP guideline update [published online Jun. 7, 2023]. Arch Pathol Lab Med. 2023.

The term “BRCAwt” as used herein means an FRα-positive cancer identified by a null mutation signature for germline BRCA mutations (gBRCAm), mutations in the BRCA1-interacting protein 1 (mBRIP1), or mutations in the DNA repair protein radiation sensitive protein 51 (mRAD51).

The term “disease control rate (DCR)” as used herein refers to the proportion of patients who achieve tumor relief (PR+CR) and stable disease after treatment and can maintain for the minimum duration requirement. Specifically, DCR includes patients with tumor maintained at stable disease: DCR=CR+PR+stable disease. The ability of a tumor to be controlled at stable disease for a long time is also one of the effective performances of drug treatment.

The term “Fc silencing mutation(s)” as used herein involves mutations in the amino acid sequence of the Fc regions of an antibody where human Fc gamma receptors of effector cells bind to an antibody. These mutations reduce or eliminate effector function while retaining binding to the neonatal Fc receptor, important for normal antibody pharmacokinetics. See, Strohl, W. R., Curr Opin Biotech 2009, 20:685-91; and Saunders, K. O., Frontiers Immunol., June 2019, vol. 10:1296.

The term “folate receptor alpha” or “FRα” as used herein is the human folate receptor alpha as encoded by the human FOLR1 gene, represented in UniprotKB as P15328.

The term “anti-folate receptor alpha antibody” or “anti-FRα antibody” or “FRα antibody” as used herein all mean an antibody that specifically binds to human folate receptor alpha. Non-limiting examples of such FRα antibodies are disclosed in, for example, WO2005/080431, WO2008/031577, WO2009/132081, WO2011/106528, WO2012/033987, WO2014/087863, WO2018/071597, WO2019/177854, WO2019/177372, WO2020/016661, WO2020/127224, WO2022/223784, WO2022/109010, WO2023/178451, WO2023/169896, WO2023/066866, WO2023/116911, among others.

The term “FRα ADC” as used herein describes a particular anti-folate receptor alpha-antibody drug conjugate having the chemical structure of Formula I: Ab-(L-D)ₚ (I), wherein Ab is an anti-FRα antibody comprising a heavy chain (HC) comprising the amino acid sequence of SEQ ID NO:3 and a light chain (LC) comprising the amino acid sequence of SEQ ID NO:4, and (L-D)ₚ has the chemical structure of Formula (II):

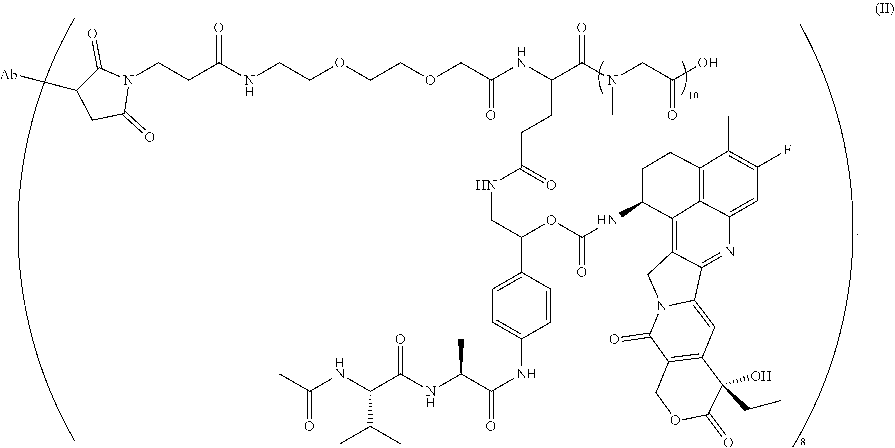

FRα ADC is also known by its generic name, sofetabart mipitecan. FRα ADC may also be found disclosed in WO2023/170247, without limitation.

The term “folate receptor alpha positive cancer” or “FRα-positive cancer” or “FRα+ cancer” as used herein means any human cancer having detectable overexpression of folate receptor alpha protein as compared to normal tissue. Detection of FRα expression may be determined by, for example, immunohistochemistry or any of the FDA-approved tests for the measurement of FRα expression available which may include, without limitation, the Ventana FOLR1 (FOLR-2.1) RxDx Assay (Ventana Medical Systems, Inc.). Non-limiting examples of FRα-positive cancers are, for example, ovarian cancer, endometrial cancer, cervical cancer, non-small cell lung cancer (NSCLC), breast cancer, triple-negative breast cancer (TNBC), pancreatic cancer, gastrointestinal cancer, renal cancer, melanoma, adenocarcinoma, brain cancers such as gliomas or glioblastomas, mesothelioma, head and neck cancer, oral squamous-cell carcinoma, and colorectal cancer (CRC). An FRα+ ovarian cancer may include, for example, epithelial ovarian cancer, primary peritoneal cancer, fallopian tube cancer, high grade serous ovarian cancer (HGSOC), high grade endometriod ovarian cancer, platinum-sensitive ovarian cancer (PSOC), platinum-resistant ovarian cancer (PROC), ovarian carcinosarcoma, uterine serous carcinoma, or ovarian clear-cell adenocarcinoma.

The term “FRα-high expression” or “high FRα expression” as used herein means a subject that suffers from an FRα-positive cancer where the level of FRα expression on the cancer is ≥75% of viable tumor cells having a moderate [2+] or strong [3+] staining intensity (≥2+ intensity) as assessed by immunohistochemistry and scored using the PS2+ scoring method. Detection of FRα expression may be determined by, for example, immunohistochemistry or any of the FDA-approved tests for the measurement of FRα expression which may include, without limitation, the Ventana FOLR1 (FOLR-2.1) RxDx Assay (Ventana Medical Systems, Inc.).

The term “FRα-moderate expression” or “moderate FRα expression” as used herein means a subject that suffers from an FRα-positive cancer where the level of FRα expression on the cancer is ≥50% to >74% of viable tumor cells having a moderate [2+] or strong [3+] staining intensity (≥2+ intensity) as assessed by immunohistochemistry and scored using the PS2+ scoring method. Detection of FRα expression may be determined by, for example, immunohistochemistry or any of the FDA-approved tests for the measurement of FRα expression which may include, without limitation, the Ventana FOLR1 (FOLR-2.1) RxDx Assay (Ventana Medical Systems, Inc.).

The term “FRα-low expression” or “low FRα expression” as used herein means a subject that suffers from an FRα-positive cancer where the level of FRα expression on the cancer is ≥25% to 49% of viable tumor cells having a moderate [2+] or strong [3+] staining intensity (≥2+ intensity) as assessed by immunohistochemistry and scored using the PS2+ scoring method. Detection of FRα expression may be determined by, for example, immunohistochemistry or any of the FDA-approved tests for the measurement of FRα expression which may include, without limitation, the Ventana FOLR1 (FOLR-2.1) RxDx Assay (Ventana Medical Systems, Inc.).

The term “FRα-very low or ultra-low expression” or “very low/ultra-low FRα expression” as used herein means a subject that suffers from an FRα-positive cancer where the level of FRα expression on the cancer is <25% of viable tumor cells having a moderate [2+] or strong [3+] staining intensity (≥2+ intensity) as assessed by immunohistochemistry and scored using the PS2+ scoring method. Detection of FRα expression may be determined by, for example, immunohistochemistry or any of the FDA-approved tests for the measurement of FRα expression which may include, without limitation, the Ventana FOLR1 (FOLR-2.1) RxDx Assay (Ventana Medical Systems, Inc.).

The term “major drug-drug interaction” as used herein means an interaction between two or more drugs, leading to unexpected side effects, such as an adverse event or reduced efficacy.

The term “metastatic” or “metastasize” as used herein means a human cancer, including the FRα-positive cancers defined herein, that spreads from the site of its origin to another part of the human body.

The term “NCI-ODWG criteria” as used herein is the abbreviation for National Cancer Institute Organ Dysfunction Working Group, and are criteria used to classify the severity of hepatic dysfunction based on serum biomarkers like aspartate aminotransferase (AST) and total bilirubin levels. For example, (a) a mild degree of hepatic dysfunction means a total bilirubin<ULN and AST>ULN, or total bilirubin>1-1.5×ULN and any AST; (b) a moderate degree of hepatic dysfunction means a total bilirubin>1.5-3×ULN and any AST; and (c) a severe degree of hepatic dysfunction means a total bilirubin>3×ULN and any AST.

The term “ocular toxicity” as used herein means a side effect in a subject's eyes after treatment with the methods of the invention. For example, ocular toxicity can encompass adverse events found in the cornea and/or retina. As non-limiting examples, ocular toxicity can be visual impairment, keratopathy, dry eye, photophobia, eye pain and/or uveitis.

The term “pulmonary toxicity” as used herein means a side effect on a subject's lungs after treatment with the methods of the invention. As a non-limiting example, pulmonary toxicity can encompass cytokine-driven interstitial lung disease (ILD).

The term “ORR is defined as the proportion of efficacy-evaluable participants who achieved a confirmed BOR of CR or PR, per RECIST v1.1. BOR is defined as the best response recorded from the start of trial treatment until the earliest of objective disease progression or start of new anticancer therapy. The corresponding 95% exact CI will be provided. DOR is defined as the time between the date of first documented response (CR or PR) to the date of first disease progression, as assessed by the Investigator per RECIST v1.1, or death due to any cause, whichever occurs first. DOR curves will be estimated using the Kaplan-Meier method. The Kaplan-Meier estimate with 95% CI calculated using Brookmeyer and Crowley method will be provided for DOR time quartiles. ORR is an index directly measuring the anti-tumor activity of a drug and is the most common endpoint based on tumor measurements.

The term “overall survival” (OS) and “median overall survival” (mOS) as used herein means a measure of the time from randomization to death used as an endpoint in clinical trials.

The term “prior PD-1/PD-L1 blockade” as used herein means any approved therapeutic that inhibits the PD-1 and/or PD-L1 pathway. Examples include, but are not limited to, pembrolizumab, dostarlimab, cemiplimab, nivolumab, atezolizumab, durvalumab or avelumab. Biosimilars of the aforementioned are also contemplated.

The term “platinum sensitive ovarian cancer (PSOC)” as used herein means an ovarian cancer that has responded to previous first-line platinum-based treatments, then recurs after at least 6 months from the last platinum-based treatment but responds to subsequent platinum-based treatment upon such recurrence.

The term “platinum resistant ovarian cancer (PROC)” as used herein means an ovarian cancer that has responded to previous first-line platinum-based treatments, then recurs after at least 6 months from the last platinum-based and/or other chemotherapy treatment but becomes resistant to such subsequent treatments.

The term “platinum-containing compound” as used herein are platinum-based antineoplastic drugs or chemotherapeutic agents that contain platinum compounds that damage DNA, used to treat cancers. Non-limiting examples include cisplatin, carboplatin and oxaliplatin.

The term “progression free survival” (PFS) and “median progression free survival” (mPFS) as used herein is defined as the time from first dose date (or randomization date for the dose optimization cohort) to the date of first documented disease progression, as assessed per RECIST v1.1, or death due to any cause, whichever occurs first. PFS curves will be estimated using the Kaplan-Meier method. The Kaplan-Meier estimate with 95% CI calculated using Brookmeyer and Crowley method.

The term “relapse” or “relapsed” as used herein is when a disease, such as a human cancer, including the folate receptor alpha positive cancers defined herein, returns following a successful treatment. A synonymous term to relapse is “recurrence”.

The term “refractory” as used herein is when a disease, such as a FRα-positive cancer defined herein, fails to respond to a medical treatment from the start of treatment. A synonymous term to refractory is “resistance”. A refractory disease, such as a refractory cancer is different from a relapsed disease, such as a relapsed cancer, defined above.

The term “serious adverse event(s)” or “SAE” is any untoward medical occurrence that at any dose: (a) results in death, (b) is life-threatening, (c) requires hospitalization or prolongation of existing hospitalization, (d) results in disability/incapacity, (e) is a congenital anomaly/birth defect, or (f) is an important medical event.

The term “TP53” as used herein is a gene in which its mutational status in human cancers, such as for example, endometrial cancers, can be linked to aggressive histological subtypes and poor prognosis as compared to wild-type (see The Cancer Genome Atlas Research Network, Nature 497, 67-73; 2013).

The term “treating” or “treat” in the context of disease as used herein refer to reducing the severity and/or frequency of one or more symptoms, eliminating one or more symptoms and/or the underlying cause of said symptoms, reducing the frequency or likelihood of one or more symptoms and/or their underlying cause, delaying, preventing and/or slowing the progression of diseases and/or disorders and improving or remediating damage caused, directly or indirectly, by the diseases and/or disorders. For clarity, treating a disease does not include and is separate from preventing a disease.

The term “treatment-emergent adverse event” (TEAE) is defined as an AE with an onset date or a pre-existing AE worsening following the first dose of trial drug through 30 days after the last dose of trial drug.

The term “time to response (TTR)” as used herein is defined as the time from first dose date (or randomization date for the dose optimization cohort) to the date of first documented response such as a complete response (CR) or a partial response (PR). TTR is only measured in participants with confirmed CR or PR.

### EXAMPLES

### Example 1: In Vivo Efficacy in Cancer Xenograft Models

Four to five-week-old female CB-17 severe combined immunodeficient (SCID) mice were obtained and quarantined for 7 days prior to study initiation. Mice were inoculated subcutaneously with 5-10×10⁶ cells of the following cancer cells resuspended in PBS: KB, a human cervical cancer cell line (ATCC CCL-17), IGROV1, a drug-resistant human ovarian carcinoma cell line (MilliporeSigma #SCC203), OV-90, a tumorigenic epithelial ovarian cancer cell line (ATCC CRL-3585), and SW-620, an adherent colorectal cancer cell line (ATCC CRL-227) or 50% BD Matrigel (Corning®) in PBS. When the average tumor volume reached about 120-150 mm³, mice were randomized (about 7 mice per group) and treated by a single intravenous injection of PBS (negative control) or 3 mg/kg FRα ADC or 3 mg/kg mirvetuximab soravtansine (Mirve sorav) as a comparator. Tumor volumes were measured every 3-5 days using a caliper device (length×width) for at least 30 days and calculated using the following formula V=4/3×π×R3, where R represents the radius. Mice were sacrificed when the tumor volume exceeded 1500 mm³ or in case of ulceration of the tumor.

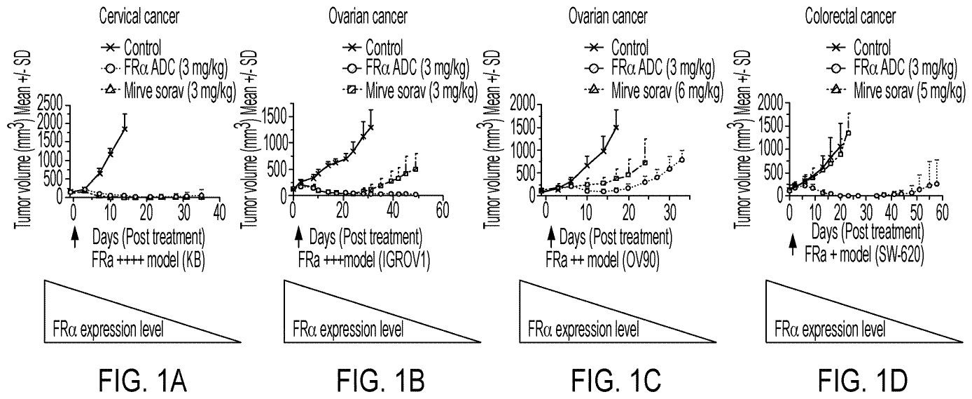

Antitumor activity was confirmed across a large collection of cell line- and patient-derived tumor xenografts with low, moderate, and high FRα expression (FIG. 1A-D). FRα ADC showed improved in vivo activity over MIRV, particularly in moderate (FIGS. 1B and 1C) and in low (FIG. 1D) FRα expressing tumor models.

### Example 2: Combination with Cisplatin and/or Bevacizumab in Ovarian Xenografts

The objective was to demonstrate the potential for enhanced activity of FRα ADC with standard-of-care agents cisplatin, bevacizumab or the combination of cisplatin plus bevacizumab, compared to FRα ADC alone in an ovarian cancer model OV-90 xenograft. Mirvetuximab soravtansine (MIRV), a folate receptor targeted ADC with a maytansanine payload was used as a comparator.

A human ovarian cancer cell line, OV-90 (CRL-3585 ATCC) were implanted unilaterally on the right flank of female immune-compromised mice (Envigo RMS, Hsd: Athymic Nude-Foxn1^{nu}) at a concentration of 5.0×10⁶ OV-90 cells). When tumor volumes reached approximately 200-300 mm³, the animals were randomized by tumor volume and body weight into control or treatment groups described in Tables 2 and 3 and dosing initiated. Dosing was discontinued after 4 weeks in Study A, after 6 weeks in Study B and tumor regrowth measurements were continued until study completion. Abbreviations in Tables 2 and 3: N/A=not applicable, IV=intravenous, IP=intraperitoneal, Q7D×4=7 times a day for 4 days.

TABLE 2
Study A - Combination of FRα
ADC with Bevacizumab and/or Cisplatin

| | | | Dose | Administration/ |
|---|---|---|---|---|
| Group | N | Treatment | (mg/kg) | Schedule |
| 1 | 5 | Vehicle (5% | N/A | IV, once |
| | | dextrose in sterile H₂0) | | |
| 2 | 5 | Bevacizumab | 5.0 | IV, once |
| 3 | 5 | Cisplatin | 4.0 | IP, Q7Dx4 |
| 4 | 5 | FRα ADC | 1.0 | IV, once |
| 5 | 5 | FRα ADC | 3.0 | IV, once |
| 6 | 5 | FRα ADC | 5.0 | IV, once |
| 7 | 5 | Mirvetuximab soravtansine | 5.0 | IV, once |
| 8 | 5 | Cisplatin | 4.0 | IP, Q7Dx4 |
| | | Bevacizumab | 5.0 | IV, once |
| 9 | 5 | FRα ADC | 1.0 | IV, once |
| | | Bevacizumab | 5.0 | IV, once |
| 10 | 5 | FRα ADC | 3.0 | IV, once |
| | | Bevacizumab | 5.0 | IV, once |
| 11 | 5 | FRα ADC | 5.0 | IV, once |
| | | Bevacizumab | 5.0 | IV, once |
| 12 | 5 | Mirvetuximab soravtansine | 5.0 | IV, once |
| | | Bevacizumab | 5.0 | IV, once |

TABLE 3
Study B - Combination of FRα
ADC with Cisplatin and/or Bevacizumab

| | | | Dose | Administration/ |
|---|---|---|---|---|
| Group | N | Treatment | (mg/kg) | Schedule |
| 1 | 4 | Vehicle (5% | N/A | IV, once |
| | | dextrose in sterile H₂0) | | |
| 2 | 4 | Cisplatin | 4.0 | IP, Q7Dx4 |
| 3 | 4 | Bevacizumab | 5.0 | IV, once |
| 4 | 4 | FRα ADC | 1.0 | IV, once |
| 5 | 4 | FRα ADC | 5.0 | IV, once |
| 6 | 4 | Mirvetuximab soravtansine | 5.0 | IV, once |
| 7 | 4 | Cisplatin | 4.0 | IP, Q7Dx4 |
| | | Bevacizumab | 5.0 | IV, once |
| 8 | 4 | FRα ADC | 1.0 | IV, once |
| | | Cisplatin | 4.0 | IP, Q7Dx4 |
| 9 | 4 | FRα ADC | 5.0 | IV, once |
| | | Cisplatin | 4.0 | IP, Q7Dx4 |
| 10 | 4 | FRα ADC | 1.0 | IV, once |
| | | Cisplatin | 4.0 | IP, Q7Dx4 |
| | | Bevacizumab | 5.0 | IV, once |
| 11 | 4 | FRα ADC | 5.0 | IV, once |
| | | Cisplatin | 4.0 | IP, Q7Dx4 |
| | | Bevacizumab | 5.0 | IV, once |
| 12 | 4 | Mirvetuximab soravtansine | 5.0 | IV, once |
| | | Cisplatin | 4.0 | IP, Q7Dx4 |
| | | Bevacizumab | 5.0 | IV, once |

Tumor volume was estimated by using the formula: v=l×w²×0.536 where l=larger of measured diameter and w=smaller of perpendicular diameter. The following termination criteria were applied to individual animals, irrespective of experimental status: (a) tumor volume >2000 mm³ (unilateral), (b) animals bearing ulcerating, skin-penetrating tumor, (c) loss of ≥20% body weight or ≥15% body weight with three consecutive measurements, and (d) severe impairment of general condition (apathy, pain, markedly reduced feed and water intake, dyspnea, abnormal habitus, or behavior).

Tumor volumes were transformed to a log 10 scale to equalize the variance across time and treatment. Log 10 volume and body weight were separately analyzed using a two-way repeated measures analysis of variance model (RM ANOVA) consisting of time, treatment, and the interaction between time and treatment using the MIXED procedure of the SAS software package (Version 9.4). Spatial Power covariance structure was used to model the correlation of observations across time for the same subject. Kenward-Roger (1997) denominator degrees of freedom (DDFM) calculations were used for tests of fixed effects. Post-hoc pairwise t-tests were used to compare tumor volumes and body weights of treated groups to the control group on the summarized day and p-values ≤0.05 were considered statistically significant.

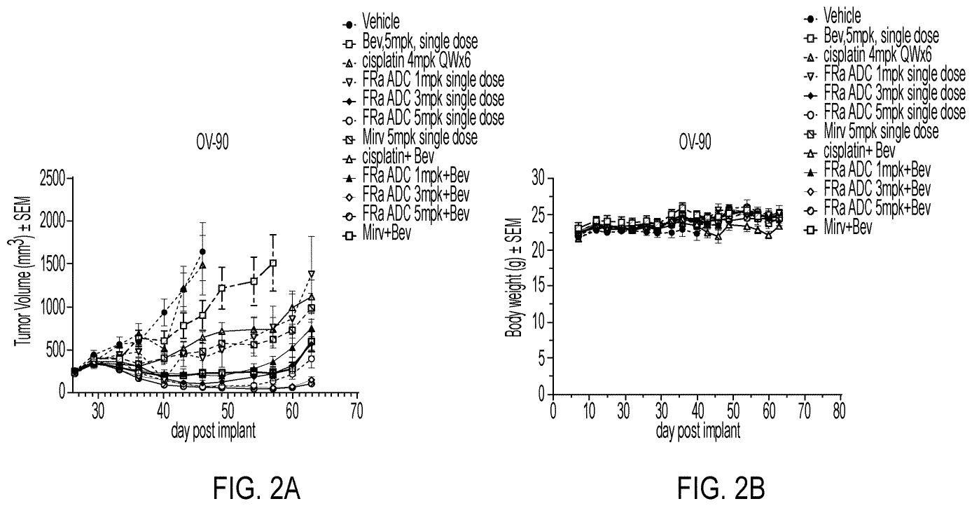

In Study A tumor growth inhibition and the combination effect analysis were calculated on Day 46. In this study FRα ADC as a single agent demonstrated dose-dependent tumor growth inhibition activity at dose levels of 1, 3 and 5 mg/kg, see FIG. 2A. The combination of FRα ADC at each of these dose levels with a single 5 mg/kg dose of bevacizumab resulted in greater anti-tumor effects than FRα ADC or bevacizumab alone with more profound effects observed at 1 and 3 mg/kg and at later time points (FIG. 2A). Additionally, when compared with standard of care therapy cisplatin plus bevacizumab, FRα ADC plus bevacizumab exhibited superior activity (FIG. 2A). Administration of FRα ADC at dose levels of 3 and 5 mg/kg led to greater anti-tumor effects than the approved comparator ADC mirvetuximab soravtansine at 5 mg/kg either as a single agent or in combination with bevacizumab (FIG. 2A).

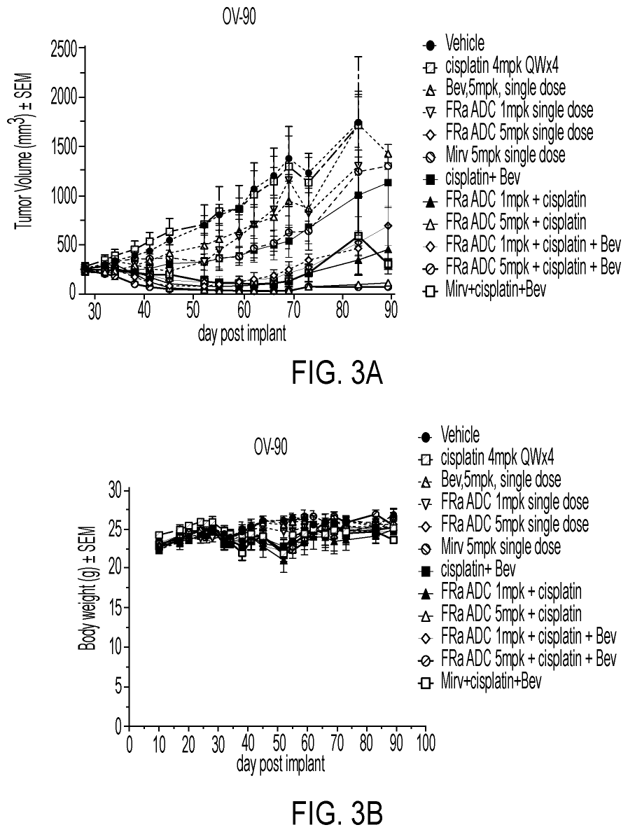

FRα ADC was further evaluated in combination with cisplatin or cisplatin plus bevacizumab in Study B. Tumor growth inhibition and combination treatment effects were determined on Days 69 and 89. The combination of FRα ADC at a single dose of 1 or 5 mg/kg with 4 mg/kg cisplatin once every 7 days showed increased activity compared to either FRα ADC or cisplatin alone (FIG. 3A). The triple combination of FRα ADC with 4 mg/kg Q7D cisplatin plus a single 5 mg/kg dose of bevacizumab also led to a marked increase of anti-tumor activity compared to the single agent treatments or to the cisplatin and bevacizumab dual combination treatment group (FIG. 3A). These increases were determined to be additive or greater than additive compared to the single agents or the standard of care combination of cisplatin and bevacizumab. Moreover, the FRα ADC 5 mg/kg triple combination treatment achieved more sustainable anti-tumor effects than either FRα ADC plus cisplatin or the combination of cisplatin and bevacizumab with the approved ADC comparator mirvetuximab soravtansine (FIG. 3A). The triplet combination regiment was also well tolerated based on the lack of significant animal weight loss observed in any of the treatment groups (FIG. 3B).

In summary, FRα ADC shows promising anti-tumor efficacy both as a single agent and when combined with standard of care therapy drugs cisplatin, bevacizumab, or cisplatin plus bevacizumab in ovarian cancer.

### Example 3: Phase 1a/1b Clinical Trial

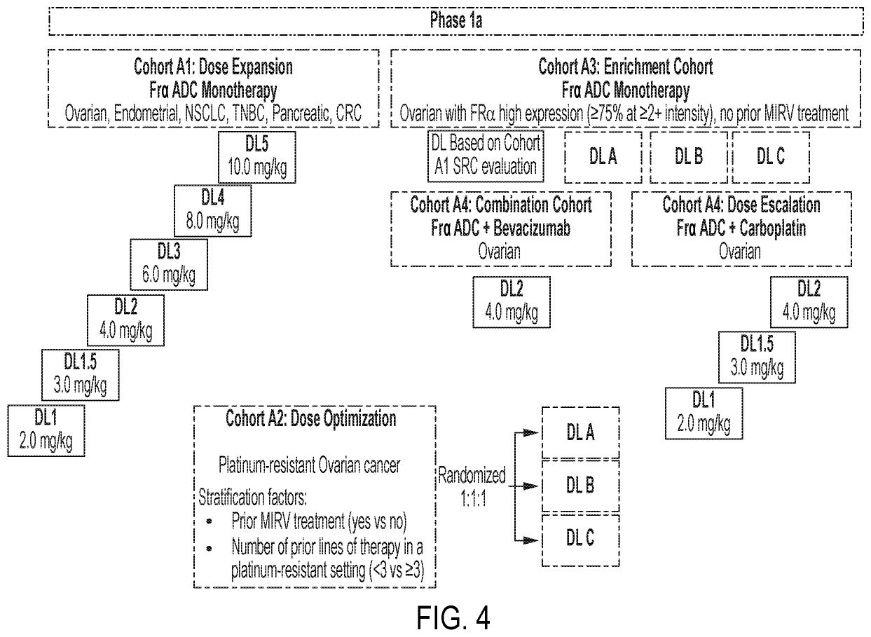

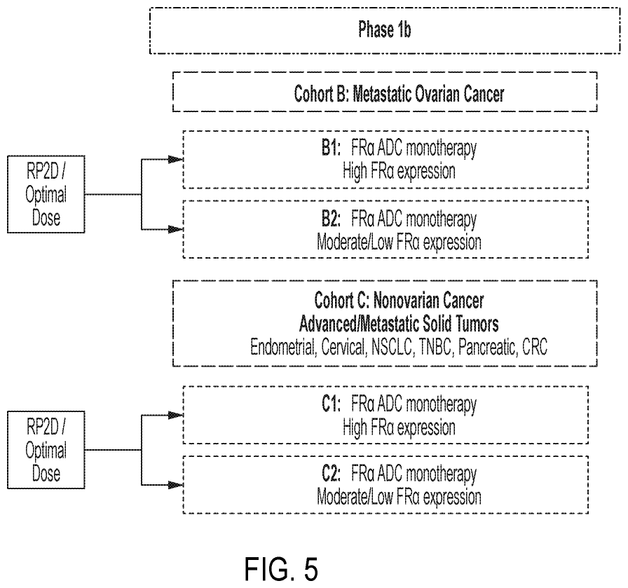

This was a Phase 1a/1b multicenter, open-label, first-in-human trial to evaluate safety, efficacy, and pharmacokinetics of FRα ADC in participants with ovarian, endometrial, cervical, NSCLC, TNBC, pancreatic, and colorectal cancers. The trial comprised 2 phases (see FIGS. 4 and 5). Phase 1a included a monotherapy dose escalation cohort (Cohort A1), a dose optimization cohort (Cohort A2), a monotherapy cohort in FRα high expressing (≥75% of viable tumors cells with at ≥2+ staining intensity) participants naive to mirvetuximab soravtansine-gynx (Elahere™; MIRV) (Cohort A3), a combination therapy cohort with bevacizumab (Cohort A4), and a combination therapy dose escalation cohort with carboplatin (Cohort A5). Dose escalation assessed safety, tolerability, PK, and preliminary efficacy of FRα ADC in participants with advanced ovarian cancer, endometrial cancer, cervical cancer, NSCLC, TNBC, pancreatic cancer, and CRC, to determine the recommended Phase 2 dose (RP2D) for Phase 1b.

To support the determination of the RP2D for Phase 1b, dose optimization (Cohort A2) further assessed safety, tolerability, PK, preliminary efficacy, and patient-reported outcomes of FRα ADC in participants with platinum-resistant ovarian cancer (progression/relapse within 6 months of last platinum dose) at 2 or more dose levels (DLs). Participants in Cohort A2 were randomized across at least 2 DLs stratified by prior MIRV treatment (yes versus no) and number of prior lines of therapy in the platinum-resistant setting (<3 versus ≥3).

In the enrichment Cohort A3, participants with platinum-resistant ovarian cancer and high FRα expression (≥75% at ≥2+ intensity) who have not been previously treated with MIRV were enrolled at the doses evaluated in Cohort A1 and deemed safe. A combination dose Cohort A4 assessed safety, tolerability, PK, and preliminary efficacy of FRα ADC in combination with bevacizumab. A dose escalation Cohort A5 assessed safety, tolerability, PK, and preliminary efficacy of FRα ADC in combination with carboplatin in participants with platinum-sensitive ovarian cancer. Approximately 160 participants were recruited for the monotherapy and combination dose escalations, approximately 20 participants were recruited for: (a) the monotherapy enrichment cohort, and (b) the combination cohort, and approximately 60 participants were recruited for the dose optimization cohort. See FIG. 4.

Phase 1b dose expansion was initiated after near completion of Phase 1a dose randomization (Cohort A2), establishing the FRα ADC RP2D/optimal dose as monotherapy. Phase 1b dose expansion included 4 dose expansion cohorts and will evaluate FRα ADC at the RP2D dose as monotherapy (see Figure x). These were participants with platinum-resistant ovarian cancer with high and moderate/low FRα expression (Cohorts B1 and B2, respectively) and a basket cohort for all other solid tumors (endometrial, cervical, NSCLC, TNBC, pancreatic, and CRC) in high and moderate/low FRα expression (Cohorts C1 and C2, respectively). FRα expression levels was determined in tumor tissue by immunohistochemistry using central testing. For Cohorts B1 and B2, available local FRα results were used for cohort allocation and confirmed. For Cohorts C1 and C2, due to the lower expression levels of FRα in the assessed histologies and lack of routine FRα testing in comparison to ovarian cancer, central testing of FRα expression levels will guide enrollment. For Cohorts C1 and C2, each histology will be capped at 5 patients. For the dose expansion cohorts, approximately 20 to 30 participants were recruited for a total of up to 100 participants.

Inclusion Criteria: Must be ≥18 years of age, or age of majority if higher per local regulations, at the time of enrollment and have historic diagnosis of locally advanced or metastatic solid tumor malignancy as defined below per specific cohort:

Cohort A1 (Dose Escalation): histological diagnosis of ovarian (epithelial ovarian, primary peritoneal, and fallopian tube) cancer, endometrial cancer, cervical cancer, NSCLC, TNBC, pancreatic cancer, or CRC.

Cohort A2 (Dose Optimization): histological diagnosis of ovarian (epithelial ovarian, primary peritoneal, and fallopian tube) cancer that is resistant to prior platinum treatment (recurrence or progression within 6 months of last platinum dose). Individuals with platinum refractory disease (i.e., progression on front-line platinum-based chemotherapy or within 3 months of completing front-line treatment) are not eligible.

Cohort A3 (Enrichment Cohort): histological diagnosis of ovarian (epithelial ovarian, primary peritoneal, and fallopian tube) cancer with high FRα expression (>75% at ≥2+ intensity), no prior treatment with MIRV, and resistant to prior platinum treatment (recurrence or progression within 6 months of last platinum dose).

Cohort A4 (Combination Cohort with bevacizumab): histological diagnosis of ovarian (epithelial ovarian, primary peritoneal, and fallopian tube) cancer that is sensitive to prior platinum treatment (CR/PR or recurrence/progression-free interval of more than 6 months of last platinum dose).

Cohort A5 (Dose Escalation combination with carboplatin): histological diagnosis of ovarian (epithelial ovarian, primary peritoneal, and fallopian tube) cancer that is sensitive to prior platinum treatment (CR/PR or recurrence/progression-free interval of more than 6 months of last platinum dose). Participants enrolled in this Cohort A5 must receive prophylactic granulocyte-colony stimulating factor (GCSF) support concurrent with the start of treatment.

Cohorts B1 and B2 (Dose Expansion): histologic diagnosis of ovarian (epithelial ovarian, primary peritoneal, and fallopian tube) cancer that is resistant to prior platinum treatment (recurrence or progression within 6 months of last platinum dose). Individuals with platinum refractory disease (i.e., progression on front-line platinum-based chemotherapy or within 3 months of completing front-line treatment) are not eligible. Must have measurable disease per RECIST v1.1.

Cohorts C1 and C2 (Dose Expansion): histologic or cytologic diagnosis of endometrial cancer, cervical cancer, NSCLC, TNBC, CRC, or pancreatic cancer. Must have measurable disease per RECIST v1.1.

Phase 1b Dose Expansion: Individuals with FRα expression levels determined by immunohistochemistry using anti-FOLR1 antibody clone FOLR1-2.1 performed in Clinical Laboratory Improvement Amendments (CLIA), International Organization for Standardization/Independent Ethics Committee (ISO/IEC), College of American Pathologists (CAP), or similarly certified laboratories as per local guidelines including, but not limited to, In Vitro Diagnostic Regulation (IVDR) compliance may enroll while central testing is pending. Must have measurable disease per RECIST v1.1. See FIG. 5.

Have an ECOG performance status of ≤1 or Karnofsky performance status of ≥80%. Have a life expectancy >12 weeks as judged by the Investigator. Must have adequate organ function, including bone marrow function, hepatic function, renal function.

Exclusion Criteria: Has a known or suspected history of active central nervous system (CNS) involvement. Has a history of carcinomatous meningitis. Has any unresolved toxicities from prior therapy. Has a serious pre-existing medical condition(s) that would preclude participation in this study, including interstitial lung disease, bowel obstruction, or severe dyspnea at rest.

Has significant cardiovascular disease, such as significant cardiovascular disease, defined as any of the following: Unstable angina or acute coronary syndrome within the past 2 months. History of myocardial infarction within 6 months prior to planned start of trial treatment. ≥Grade 3 New York Heart Association functional classification system of heart failure, uncontrolled or symptomatic arrhythmias. ≥Grade 3 New York Heart Association functional classification system of heart failure, uncontrolled or symptomatic arrhythmias.

Has prolongation of the corrected QT interval by Fridericia (QTcF)>470 milliseconds during Screening. QTcF is calculated using Fridericia's Formula: QTcF=QT/(RR^{0.33}).

Has the following known infections: active uncontrolled systemic bacterial, viral, fungal, or parasitic infection, active hepatitis B virus (HBV) Participants who are HBsAg positive and HBV DNA positive (detectable) will be excluded, active hepatitis C virus (HCV), active HIV infection, active cytomegalovirus infection.

Has active second malignancy unless in remission with life expectancy >2 years.

Cohorts A2 (Dose Optimization), and B1 and B2 (Dose Expansion): has ovarian cancer with mucinous or clear cell histologies.

Cohorts A2 (Dose Optimization), and B1 and B2 (Dose Expansion): Has ovarian cancer with hypoalbuminemia (≤3.5 g/dL), malnutrition and weight loss (Grade ≥2), or presence of ascites that requires placement of indwelling drainage catheters or frequent draining procedures (more than 2 times in the last 28 days).

Has received Prior/Concomitant Therapy therapies Cytotoxic therapies: ≤14 days or ≤5 half-lives, whichever is shorter. Has received therapeutic monoclonal antibodies: ≤14 days. Has received radiotherapy: ≤7 days or ≤21 days. Has received major surgery: ≤28 days.

Is on an interventional, anticancer therapeutic trial in the treatment phase. Has prior treatment with a FRα ADC with a topoisomerase I inhibitor.

Cohorts A2 (Dose Optimization), and Bland B2 (Dose Expansion): Has received more than 4 prior lines of systemic treatment in the platinum-resistant setting.

Tumor Measurements: Tumors will be assessed in accordance with the SOA and the Imaging Manual. Assessments of both measurable and nonmeasurable disease as assessed by using RECIST v1.1 as appropriate to tumor type, or a comparable assessment method depending on the location of the tumor.

Response Assessment: For all participants, imaging studies (CT, including spiral CT, or MRI scan of the chest, abdomen, and pelvis) will be performed locally at baseline. It is recommended that CT imaging of the abdomen and pelvis be performed with IV contrast, whenever possible. The CT portion of a PET-CT scan may be used as a method of response assessment if the site can document that the CT is of identical diagnostic quality to a diagnostic CT (with IV and oral contrast). A PET scan alone or as part of a PET-CT may be performed as part of routine clinical care but cannot be used to assess response according to RECIST v1.1.

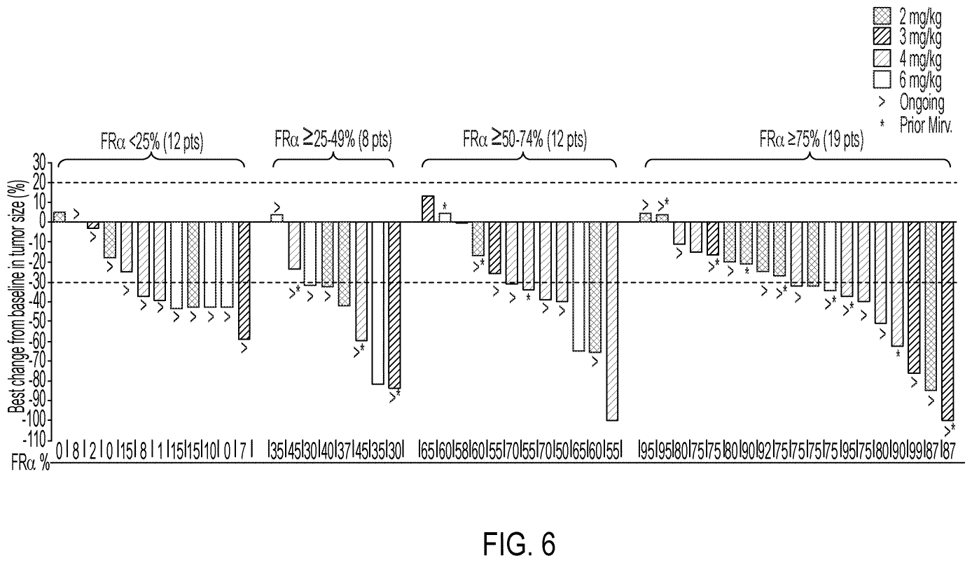

Summary of results: Early clinical data is in line with pre-clinical findings where FRα ADC was well-tolerated and demonstrated activity in HGSOC patients, including those with FRα expression levels <75% and in MIRV pre-treated patients. See FIG. 6. Specifically, no ocular or pulmonary toxicity and no neuropathy was observed. Table 4 summarizes the assessed HGSOC patient complete responses (CR)/partial responses (PR), separated by FRα expression levels and whether or not there was prior MIRV treatment.

TABLE 4
FRα ADC Monotherapy in High-Grade Serous Ovarian Cancer

| | FRα ≥75% | FRα ≥75% | | |
|---|---|---|---|---|
| | MIRV Naïve | MIRV Treated | FRα <75% | Total |
| Responses | (N = 11) | (N = 8) | (N = 34) | (N = 53) |
| CR/PR | 6 (54.5) | 4 (50.0) | 16 (47.1) | 26 (49.1) |
| (confirmed or | | | | |
| pending | | | | |
| confirmation) | | | | |

Table 4A summarizes the overall response rate (ORR) for 104 efficacy evaluable high grade serous ovarian cancer patients from the Phase I dose escalation (Cohort A1) and dose optimization (Cohort A2).

TABLE 4A
FRα ADC Monotherapy in High-Grade Serous Ovarian Cancer

| | | | Prior MIRV |
|---|---|---|---|
| Responses | FRα ≥75% | FRα <75% | treatment |
| ORR n/N (%) | 26/46 (57%) | 23/53 (43%) | 11/18 (61%) |

From Table 4A, it was observed that FRα ADC was well tolerated and resulted in durable clinical efficacy in heavily pretreated patients with HGSOC, regardless of FRα expression level or prior MIRV treatment.

Table 5 below: efficacy evaluable patients are those who had at least 1 post-baseline response assessment or had discontinued treatment before the first post-baseline response assessment. Patients in both dose escalation and dose optimization cohorts are shown. The ORR in Table 5 includes patients with unconfirmed and confirmed CR and PR.

TABLE 5
Anti-Tumor Activity by Dose Level Q3W - HGSOC

| Efficacy | | | | | |
|---|---|---|---|---|---|
| Evaluable | 2 mg/kg | 3 mg/kg | 4 mg/kg | 6 mg/kg | Total |
| Patients | (N = 15) | (N = 10) | (N = 18) | (N = 11) | (N = 54) |
| ORR, % (n/N) | 33 (5/15) | 40 (4/10) | 61 (11/18) | 55 (6/11) | 48 (26/54) |
| CR, n (%) | — | — | 1 (6) | 1 (9) | 2 (4) |
| PR, n (%) | 5 (33) | 4 (40) | 10 (55) | 5 (45) | 24 (44) |
| DCR, % (n/N) | 80 (12/15) | 80 (8/10) | 83 (15/18) | 73 (8/11) | 81 (44/54) |

Summary: As shown in Table 5, FRα ADC showed promising efficacy in patients with HGSOC across all dose levels.

Table 5A below shows an updated data analysis of efficacy evaluable 106 PROC patients with measurable disease at baseline who received at least one dose of FRα ADC. uORR=(CR+unconfirmed CR+PR+unconfirmed PR) and cORR=(CR+PR) and DCR=(CR+uCR+PR+uPR+stable disease).

TABLE 5A
Anti-Tumor Activity by Dose Level Q3W - PROC

| Efficacy | | | | | |
|---|---|---|---|---|---|
| Evaluable | 2 mg/kg | 3 mg/kg | 4 mg/kg | 6 mg/kg | Total |
| Patients | (N = 35) | (N = 16) | (N = 37) | (N = 14) | (N = 102) |
| uORR, % (n) | 37.1 (13) | 68.8 (11) | 56.8 (21) | 50.0 (7) | 51.0 (52) |
| cORR, % (n) | 22.9 (8) | 43.8 (7) | 45.9 (17) | 50.0 (7) | 38.2 (39) |
| DCR, % (n) | 74.3 (26) | 87.5 (14) | 75.7 (28) | 71.4 (10) | 76.5 (78) |

Summary: In Table 5A, FRα ADC demonstrated clinically meaningful efficacy in heavily pretreated patients with PROC. Overall, tumor responses were observed across all doses evaluated with the highest uORR/cORR and DCR observed for the 3 mg/kg and 4 mg/kg doses. Notably, tumor responses were observed regardless of FRα expression levels and in participants previously treated with MIRV.

A patient subgroup evaluation was done on forty-seven (47) patients aged 65 and over with PROC who had a median of 4 prior lines of therapy, including prior treatment with MIRV, and with an FRα expression of <75% were treated with 2, 3, 4, or 6 mg/kg of FRα ADC. For this patient subgroup, the ORR was 51%. Separately, another evaluation was done on seventeen (17) patients with PSOC aged 65 and over were treated with 4 mg/kg of FRα ADC in combination with bevacizumab. These patients had a median of 2 prior lines of therapy, including prior treatment with MIRV, and an FRα expression of <75%. For this patient subgroup, the ORR was 29%. These combined patient subgroup data in PROC and PSOC demonstrate promising efficacy of FRα ADC in adults aged 65 and over, who comprise nearly half of all patients diagnosed with ovarian cancer.

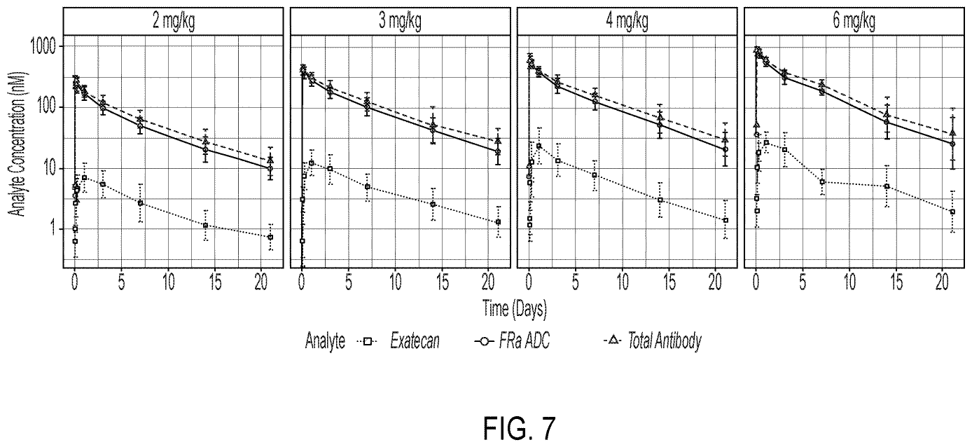

The plasma concentration of FRα ADC, total antibody and total unconjugated exatecan after Cycle 1 was measured at doses tested: 2 mg/kg, 3 mg/kg, 4 mg/kg and 6 mg/kg. See FIG. 7, where the data shown are geometric means and standard deviation (SD). This shows that FRα ADC concentration in plasma was stable with minimal accumulation prior to the subsequent doses (gomean half-life of payload 6.5-8 days). Table 6 summarizes the comparison of FRα ADC and exatecan payload PK parameters.

TABLE 6
FRα ADC and Payload Summary PK

| Dose | AUC₀₋ₗₐₛₜ (ng*day/mL) | | Cmax (ng/mL) | | Half-life (days) | |
|---|---|---|---|---|---|---|
| (mg/kg) | FRα ADC | Exatecan | FRα ADC | Exatecan | FRα ADC | Exatecan |
| 2 | 4440000 (30) | 519 (63) | 45500 (22) | 3.3 (62) | 5.6 (2.3-9.3) | 7.9 (5.3-8.0) |
| 3 | 8060000 (27) | 1110 (64) | 73200 (21) | 5.9 (64) | 5.5 (4.3-7.5) | 7.6 (5.1-7.6) |
| 4 | 10400000 (23) | 1580 (66) | 104000 (15) | 9.1 (78) | 5.5 (3.3-7.7) | 6.5 (2.5-10.3) |
| 6 | 14800000 (36) | 1780 (60) | 143000 (18) | 10 (56) | 5.2 (3.1-7.1) | 7.1 (5.0-10.5) |

Table 6 demonstrates that the PK of FRα ADC and exatecan was dose proportional from 2 mg/kg to 6 mg/kg. The overall PK profile of FRα ADC supports dosing at Q3W.

Table 7 shows the Phase I efficacy results, as calculated by ORR %, broken down by the patient's tumor FRα expression levels, if known.

TABLE 7
Efficacy by Tumor FRα expression levels

| | | | | | FRα |
|---|---|---|---|---|---|
| | | FRα <75% | | | not |
| | FRα ≥75% | 0-24% | 25-49% | 50-74% | known |
| ORR % | 42% (11/26), | 23% | 39% | 38% | 8% |
| (n/N) | includes 19% | (3/13) | (5/13) | (5/13) | (2/26) |
| | (5/16) pts with | | | | |
| | prior MIRV | | | | |

The results demonstrate that FRα ADC has clinical activity across all FRα expression levels regardless of prior MIRV treatment.

### Example 4: Translational PK/PD Modeling

Understanding FRα ADC's PK/PD characteristics is crucial to optimizing its therapeutic window and predicting clinical outcomes. This study assessed how PK and efficacy endpoints in preclinical experiments of FRα ADC translates into human patients.

FRα ADC non-human primate PK data were modelled using a two-compartment model for total antibody/conjugated ADC and a one-compartment model for exatecan. Human PK was predicted using body-weight-based allometric scaling from non-human primate and used to estimate the minimum efficacious dose required to match the exposure needed for >80% tumor regression in xenograft and PDX-bearing Tg32 mice, such as those demonstrated in Examples 1 and 2. Prediction accuracy of the human PK/efficacy model was assessed by comparing it to observed clinical data, such as those described in Example 3.

The preclinical PK model accurately predicted the human PK and therapeutic index of FRα ADC and unconjugated payload within 0.8 to 1.25-fold (see Table 8). Slower deconjugation of exatecan in patients resulted in lower plasma payload levels and prolonged FRα ADC stability, aligning the PSAR linker platform DAR stability results between preclinical and clinical studies. Preclinical and emerging clinical PKPD data suggested a broader therapeutic index for FRα ADC, supporting clinical efficacy and safety across a 2-3× dose range from the Phase I trial (Example 3).

TABLE 8
Comparison of Predicted Human PK versus Observed PK

| | Predicted Human PK based on | | Observed PK from Patients (N = 21) | |
|---|---|---|---|---|
| | preclinical PK/PD Modeling | | AUC (ng day/mL) ± | Cmax (ng/mL) ± |
| | AUC (ng day/mL) | Cmax (ng/mL) | SD | SD |
| Unconjugated | 17.7 | 3.07 | 21.6 ± 14.9 | 3.28 ± 2.84 |
| Exatecan Payload | | | | |

Preclinical evaluation combined with translational modelling of PSAR-linked exatecan ADC provided critical data on payload release rate, half-life of FRα ADC (and its components), a projected efficacious dose and dosing regimen with human PK/PD characteristics to support the Phase I trial design of FRα ADC.

### Example 5: Phase 3 Clinical Trials

This Phase 3 study aims to evaluate FRα ADC for the treatment of patients with advanced ovarian cancer, which includes high grade serous ovarian (HGSOC), high grade endometriod ovarian, primary peritoneal, and fallopian tube cancers. The study comprises 3 independent parts, each addressing different patient groups: Part A: platinum-resistant ovarian cancer (PROC), Part B: platinum-sensitive ovarian cancer (PSOC), and Part C: PSOC who are PARP inhibitor naive. The primary endpoint for Part A, Part B and Part C is investigator-assessed progression-free survival (PFS) per RECIST v1.1. A key secondary endpoint is assessing efficacy as measured by overall survival (OS) of: (a) FRα ADC monotherapy versus control in Part A or (b) FRα ADC plus bevacizumab versus control in Parts B and C.

Part A: Evaluation of FRα ADC as monotherapy at 3 mg/kg versus a control arm of a choice of paclitaxel at 80 mg/m², pegylated liposomal doxorubicin (PLD) at 40 mg/m², gemcitabine at 800-1000 mg/m², topotecan at 4 mg/m², or MIRV at 6 mg/kg AIBW (for MIRV-naive participants with FRα positivity as per the local product label [≥75% viable tumor cells with ≥2+ staining intensity], where approved and commercially available). See Table 9.

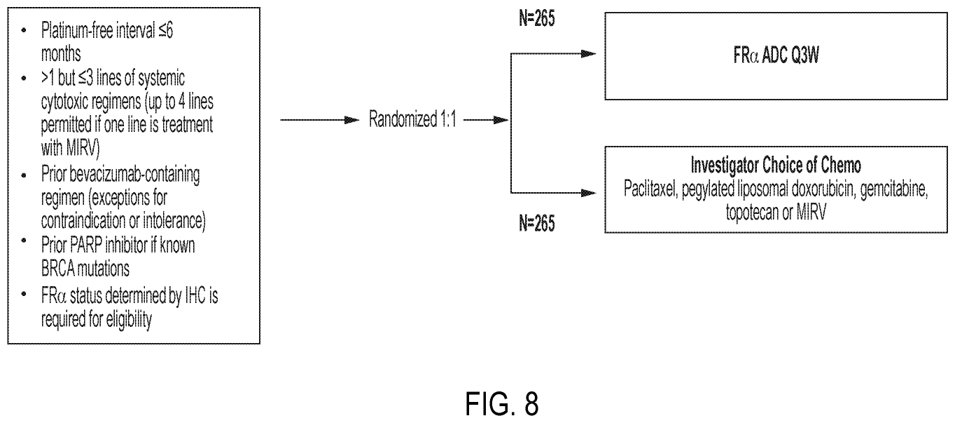

Participants with PROC, defined as radiographic progression ≤6 months of the last administration of platinum therapy, who have undergone up to 3 prior lines of systemic cytotoxic therapy (or up to 4 if MIRV was included) will be enrolled in Part A. Participants will be randomly assigned in a 1:1 ratio to receive FRα ADC or control. Randomization of participants will be stratified as follows: (a) high FRα expression (≥75% viable tumor cells with ≥2+ staining intensity): yes versus no; (b) prior MIRV use: yes versus no; and (c) investigator's choice of therapy: paclitaxel versus other chemotherapy versus MIRV. See FIG. 8.

Exclusion criteria for Part A (PROC) may include the following types of patients:
1. Have received more than 4 prior lines of systemic cytotoxic therapy:
   a. Neoadjuvant+/−adjuvant therapies are considered 1 line of therapy if the neoadjuvant or adjuvant corresponds to 1 fully predefined regimen.
   b. Maintenance therapies are considered as part of the preceding line of therapy and not counted independently.
   c. Change of neoplastic agents due to toxicity are not considered a new line of therapy.
   d. If MIRV was not included in the previous lines, only 3 prior systemic cytotoxic therapy lines are allowed.
2. Have primary platinum-refractory disease defined as disease that progressed ≤3 months since the last dose of first-line platinum-containing chemotherapy.
3. Have received prior ADC with a topoisomerase inhibitor payload.

Part B: Evaluation of FRα ADC at 3 mg/kg in combination with bevacizumab at 15 mg/kg versus a control arm of a choice of a platinum-based doublet chemotherapy: (a) carboplatin at AUC 5-6 mg/mL/min and paclitaxel at 175 mg/m² plus bevacizumab at 15 mg/kg; (b) carboplatin at AUC 4 mg/mL/min and gemcitabine at 800-1000 mg/m² plus bevacizumab at 10 mg/kg, or carboplatin/pegylated liposomal doxorubicin) plus bevacizumab at 10 mg/kg, in participants with PSOC. See Table 10.

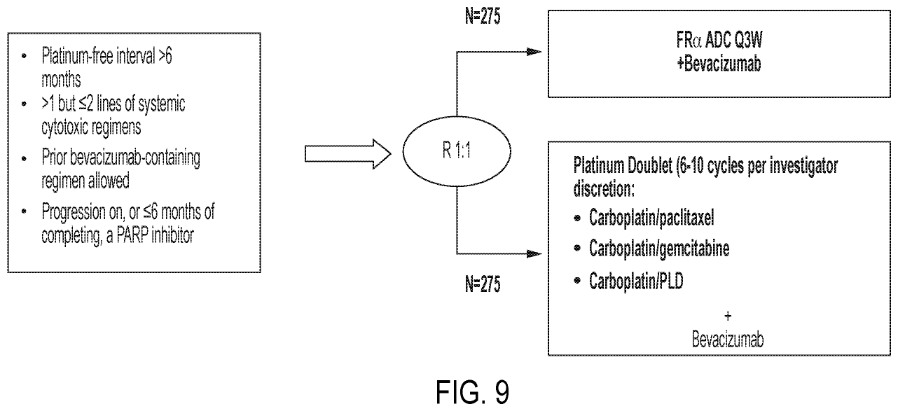

Participants with PSOC who have undergone up to 2 prior lines of systemic cytotoxic therapy and progressed either on or within 6 months of completing treatment with a poly (ADP-ribose) polymerase (PARP) inhibitor will be enrolled in Part B. Participants will be randomly assigned in a 1:1 ratio to receive FRα ADC plus bevacizumab or investigator's choice of platinum-based doublet chemotherapy: (a) carboplatin at AUC 5-6 mg/mL/min and paclitaxel at 175 mg/m² plus bevacizumab at 15 mg/kg; (b) carboplatin at AUC 4 mg/mL/min and gemcitabine at 800-1000 mg/m² plus bevacizumab at 10 mg/kg; or (c) carboplatin at AUC 5 mg/mL/min and pegylated liposomal doxorubicin at 30 mg/m² plus bevacizumab at 10 mg/kg. Randomization of participants will be stratified as follows: (a) platinum-free interval: <12 versus >12 months; (b) disease recurrence: first versus second; and (c) investigator's choice of carboplatin-based doublet: carboplatin plus either paclitaxel versus gemcitabine versus pegylated liposomal doxorubicin (PLD). See FIG. 9.

Exclusion criteria for Part B (PSOC) may include the following types of patients:
1. Have received more than 2 prior lines of systemic cytotoxic therapy. Lines of prior antineoplastic regimens are counted according to the following descriptions:
   a. Neoadjuvant+/−adjuvant therapies are considered 1 line of therapy if the neoadjuvant or adjuvant corresponds to 1 fully predefined regimen.
   b. Maintenance therapies are considered as part of the preceding line of therapy and not counted independently.
   c. Change of neoplastic agents due to toxicity are not considered a new line of therapy.
2. Have clinically significant proteinuria: if a urine dipstick result ≥3+, participant should complete 24-hour urine collection and must show result ≤1 g of protein in a 24-hour period to be eligible.
3. Have received prior ADC with a topoisomerase inhibitor payload.

Administration of FRα ADC in both Parts A and B will be by intravenous (IV) infusion on Day 1 of 21-day cycles (Q3W). Doses will be based on actual body weight on Day 1 of each cycle. Administration of FRα ADC should be prior to administration of bevacizumab in Part B. Bevacizumab will be administered per product label on Day 1 of 21-day cycles (Q3W) by IV. See Tables 9 and 10.

TABLE 9
Part A (PROC)

| Experimental | Control Arm | | | | |
|---|---|---|---|---|---|
| FRα ADC | Paclitaxel | Topotecan | Gemcitabine | PLD | MIRV |
| 3 mg/kg | 80 mg/m² | 4 mg/m² | 800-1000 | 40 mg/m² | 6 mg/kg |
| | | | mg/m² | | AIBW |
| Day 1 | Days 1, 8, 15 | Days 1, 8 and | Days 1 and 8 | Day 1 | Day 1 |
| | and 22 | 15 | | | |
| 21 day cycle | 28 day cycle | 28 day cycle | 21 day cycle | 28 day cycle | 21 day cycle |
| IV route | IV route | IV route | IV route | IV route | IV route |

TABLE 10
Part B and Part C (PSOC)

| | Control Arm | | | |
|---|---|---|---|---|
| Experimental | Carboplatin and | Carboplatin and | | |
| FRα ADC + | Paclitaxel plus | Gemcitabine plus | Carboplatin and | Bevacizumab |
| Bevacizumab | Bevacizumab | Bevacizumab | PLD | (maintenance) |
| 3 mg/kg + | AUC 5-6 | AUC 4 mg/mL/min | AU 5 mg/mL/min | 15 mg/kg |
| 15 mg/kg | mg/mL/min and | and 800-1000 | and 30 mg/m2 plus | |
| | 175 mg/m2 plus | mg/m2 plus 10 | 10 mg/kg | |
| | 15 mg/kg | mg/kg | | |
| Day 1 | Day 1 | Day 1 and | Day 1 and Day 1 | Day 1 |
| | | Days 1 and 8 plus | plus Days 1 and 15 | |
| | | Day 1 | | |
| 21 day cycle | 21 day cycle plus | 21 day cycle plus | 28 day cycle plus | 21 day cycle |
| | 21 days | 21 days | 28 days | |
| IV route | IV route | IV route | IV route | IV route |

A Part C will be added to the Phase 3 plan comprising approximately 550 patients with PSOC who are PARP inhibitor naïve to compare the efficacy of FRα ADC at 3 mg/kg in combination with bevacizumab at 15 mg/kg or its biosimilar in this patient group with a control arm: investigator's choice of a platinum, carboplatin doublet: (i) carboplatin/paclitaxel, (ii) carboplatin/gemcitabine, or (iii) carboplatin/pegylated liposomal doxorubicin, each in combination with bevacizumab or its biosimilar. Participants with PSOC who have undergone up to 2 prior lines of systemic cytotoxic therapy will be enrolled in Part C. The control arm of Part C includes the following platinum doublet regimen selected by the investigator prior to randomization: (i) carboplatin/paclitaxel, (ii) carboplatin/gemcitabine, or (iii) carboplatin/pegylated liposomal doxorubicin in combination with bevacizumab or its biosimilar. Randomization 1:1 ratio of participants in Part C will be stratified as follows: (a) high FRα expression (≥75% of cells with ≥2+ staining intensity)—yes versus no; (b) platinum-free interval: <12 months versus>12 months; (c) disease recurrence—first versus second; and (d) investigator's choice of platinum, carboplatin doublet: (i) carboplatin/paclitaxel, (ii) carboplatin/gemcitabine, or (iii) carboplatin/pegylated liposomal doxorubicin.

Inclusion criteria for Part C—PSOC: (a) must have relapsed after first-line platinum-based chemotherapy and have platinum-sensitive disease defined as radiographic progression >6 months after their last administration of platinum therapy, (b) must be a candidate for subsequent treatment with a platinum doublet, as defined above, and with bevacizumab or its biosimilar; and (c) must have previously received >1 but <2 prior lines of systemic cytotoxic chemotherapy.

Exclusion criteria for Part C—PSOC: (a) have received more than 2 prior lines of systemic cytotoxic chemotherapy; (b) have previously received treatment with a PARP inhibitor; and (c) have clinically significant proteinuria: if urine dipstick result >3+, participant should complete 24-hour urine collection and must show result ≤1 g of protein in a 24-hour period to be eligible.

For Part A PROC and Parts B and C PSOC, FRα ADC will be administered until radiographic progression, unacceptable toxicity or other discontinuation criteria are met.

### Example 6: Phase Ia Dose Optimization Combination in Endometrial Cancers

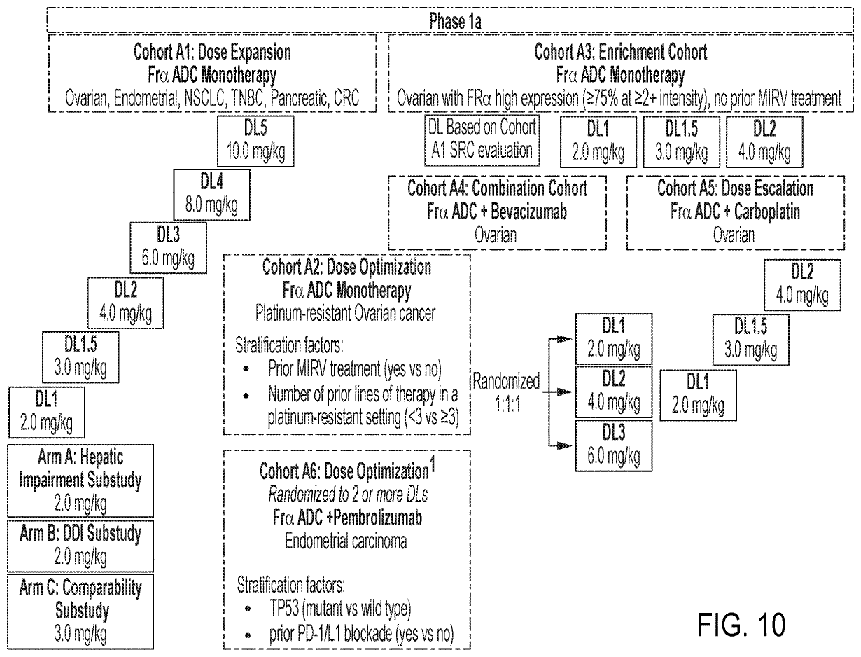

As an addendum to the Phase Ia disclosed in Example 3, the optimal dose of the combination of FRα ADC plus pembrolizumab and its anti-tumor activity will be examined in participants with endometrial cancers in newly added Cohort A6. Approximately 60 participants in Cohort A6 will be randomized across at least 2 dose levels and stratified by TP53 status (mutant versus wild type) and prior PD-1/PD-L1 blockade (yes versus no). FRα ADC will be administered first, followed by pembrolizumab. FRα ADC will be administered at 3 mg/kg and at 4 mg/kg. Pembrolizumab will be administered per product package label on Day 1 of 21-day cycles (Q6W) intravenously (IV). Pembrolizumab will be given for up to 2 years or until disease progression. See FIG. 10.

Inclusion criteria for Cohort A6 can include but is not limited to the following: (a) a histological diagnosis of mismatch repair-proficient (pMMR)/microsatellite stable (MSS) endometrial carcinoma that has progressed within 12 months of last dose of platinum-taxane chemotherapy, and (b) participants must have measurable disease per RECIST v1.1.

Exclusion criteria for Cohort A6 can include, but is not limited to the following: (a) participants receiving doses of prednisone (or its equivalent) higher than 10 mg/day are ineligible to enroll; (b) participants having endometrial stromal sarcoma, leiomyosarcoma or adenosarcoma are ineligible; and (c) participants deemed not appropriate to receive pembrolizumab per the treating physician, e.g., autoimmune disease, history of ILD or pneumonitis, etc.

### Example 7: Phase Ia Substudies

In the Phase Ia study described in Example 3, new substudies in the dose escalation Cohort A1 will be conducted to evaluate the safety and PK properties of FRα ADC in participants: (a) with moderate hepatic impairment (Arm A), (b) in a drug-drug interaction (DDI) study with a strong CYP3A4 inhibitor (Arm B), and (c) in an early-phase versus late-phase formulation comparability evaluation (Arm C). See FIG. 10.

Cohort A1, Arm A: Between 8 to 10 eligible participants with gynecological cancers, such as, for example ovarian, cervical, endometrial, etc., and with moderate hepatic impairment (a total bilirubin >1.5 to 3× upper limit of normal (ULN) and any aspartate aminotransferase (AST), based on NCI-ODWG criteria) will be evaluated for safety and PK of FRα ADC monotherapy treatment at 2 mg/kg. The analysis will use a population PK model to assess hepatic function as a covariate on PK parameters, including but not limited to AUC and Cmax. Inclusion criteria for Arm A can include a histological diagnosis of gynecological cancer, which can include, for example, ovarian cancer, endometrial cancer, cervical cancer, with moderate hepatic impairment. The acceptable hepatic function for Arm A uses the NCI-ODWG criteria wherein total bilirubin is >1.5 to 3× the upper limit of normal (ULN), and AST can be any AST.

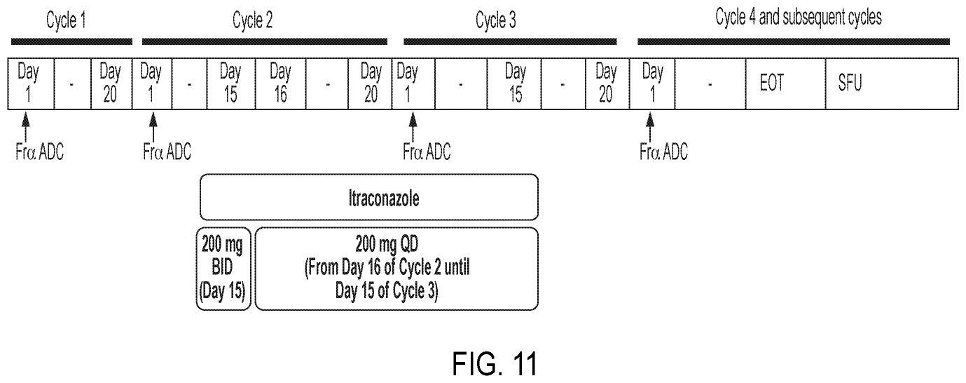

Cohort A1, Arm B: A DDI study for FRα ADC with itraconazole will be conducted in participants with gynecological cancers (ovarian, cervical, endometrial, etc). Itraconazole will be administered according to its product label. The substudy is an open-label fixed-sequence substudy designed to evaluate the effect of itraconazole, a strong CYP3A4 inhibitor, on the PK and safety of the exatecan payload and FRα ADC in eligible participants. The substudy will enroll approximately 12 to 16 participants, and include the treatment periods in Cycles 2 and 3: (a) in Cycle 2, the participants will receive a single dose of 2 mg/kg FRα ADC on Day 1, then on Day 15, the participants will receive itraconazole at a dose of 200 mg by mouth twice daily, once at the clinic and once at-home; (b) starting from Day 16 of Cycle 2, the participants will take 200 mg once daily by mouth at-home through Day 15 of Cycle 3 (for a total of approximately 21 days); and (c) on Day 1 of Cycle 3, the participants will receive a single dose of 2 mg/kg of FRα ADC. See FIG. 11. Blood samples for PK analysis on exatecan payload including but not limited to AUC and Cmax will be collected at specified time points. PK samples of itraconazole and hydroxy-itraconazole will be collected to ensure a steady-state concentration of itraconazole. Safety and tolerability, including AEs, vital signs, ECGs, and laboratory parameters will be monitored throughout the substudy, including weekly labs during Cycle 3. Beginning at Cycle 4, FRα ADC dose may be increased after discussion and approval by medical monitor, if the treating physician feels that the participant would benefit from a dose increase. Inclusion criteria for Arm B can include a histological diagnosis of gynecological carcinoma, which can include, for example ovarian cancer, endometrial cancer, or cervical cancer. Exclusion criteria can include: (a) those individuals unable to receive itraconazole orally or with any medical conditions, medical history, or are taking any medications that are contraindicated in the itraconazole prescribing information; and (b) those individuals with hearing loss that requires the use of assistive devices, such as hearing aids.

Cohort A1, Arm C: To support the transition from early-phase to late-phase, an appropriate comparability assessment will be performed to demonstrate that the pre-change and post-change clinical materials do not demonstrate any clinically meaningful differences in product quality and safety profile. Pharmacokinetics in participants treated with the early-phase material (Dose Escalation Cohort A1 and Dose Optimization Cohort A2) or the late-phase material (Cohort A1: Arm C substudy) at 3 mg/kg will be compared. Approximately 12 to 16 participants with ovarian cancer, cervical cancer, endometrial cancer, NSCLC, and TNBC will be enrolled into Cohort A1: Arm C. Non-compartmental or pop-PK analysis with AUC and Cmax as PK parameters of FRα ADC will be used to demonstrate the comparability of the early-phase and late-phase clinical materials. If necessary, the propensity score matching approach will also be considered to balance the disease characteristics between these two groups. Inclusion criteria can include a histological diagnosis of ovarian cancer, endometrial cancer, cervical cancer, non-small cell lung cancer, and triple negative breast cancer.

### Example 8: Phase Ib Dose Expansion

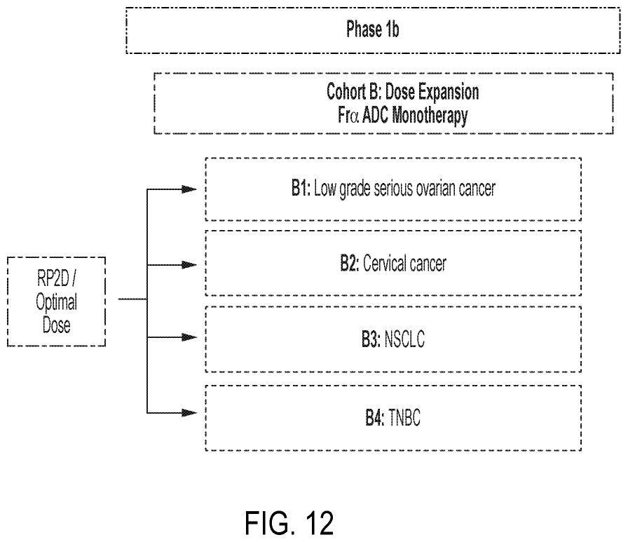

As an amendment to the Phase Ib described in Example 3, additional cohorts will be added to Cohort B. This study will evaluate FRα ADC monotherapy in participants with low grade serous ovarian cancer, with cervical cancer, with NSCLC, and with TNBC where each cohort will be treated with FRα ADC at the recommended phase 2 dose (RP2D) as determined by the Phase Ia. See FIG. 12. Up to approximately 100 eligible participants will be added to four new cohorts with approximately 25 participants each:

Cohort B1 dose expansion will enroll eligible participants with histologic or cytologic diagnosis of low grade ovarian serous ovarian cancer and measurable disease per RECIST v1.1 who have received prior platinum containing treatment.

Cohort B2 dose expansion will enroll eligible participants with histologic diagnosis of squamous cell, adenocarcinoma, glassy cell, or adenosquamous cervical cancer and measurable disease per RECIST v1.1. Participants must have received prior platinum containing chemotherapy.

Cohort B3 dose expansion will enroll eligible participants with histologic or cytologic diagnosis of lung adenocarcinoma and measurable disease per RECIST v1.1, in which participants must have received prior PD-1/PD-L1 inhibitor therapy; participants with activation oncogenic mutations must have received approved mutation based thymidine kinase inhibitor (TKI) therapy; and prior PD-1/PD-L1 is not a requirement for participants with oncogenic drivers.

Cohort B4 dose expansion will enroll eligible participants with histologic diagnosis of triple negative breast cancer (TNBC) and measurable disease per RECIST v1.1. Triple negative receptor status is defined as ≤10% for estrogen and progesterone receptor expression and human epidermal growth factor receptor 2 (HER2) negative per ASCO/CAP HER2 testing guideline.

1. A method of treating a subject having a folate receptor alpha-positive cancer, the method comprising administering an anti-folate receptor alpha-antibody drug conjugate in a dosing regimen comprising: a dose from about 1.0 mg/kg to about 10.0 mg/kg dose as an intravenous (IV) infusion on Day 1 of every 21-day cycle (Q3W) for at least 2 cycles, wherein the anti-folate receptor alpha-antibody drug conjugate has the chemical structure of Formula II: 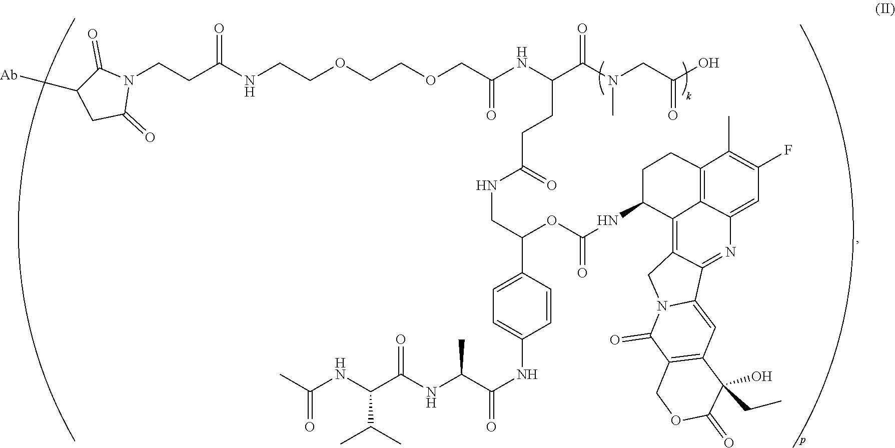
wherein Ab is an anti-FRα antibody, k is from 2-20 and p is from 1-8.

2. The method of claim 1, wherein the anti-FRα antibody comprises a heavy chain (HC) comprising the amino acid sequence of SEQ ID NO:3 and a light chain (LC) comprising the amino acid sequence of SEQ ID NO:4.

3. The method of claim 2, wherein k is 10 and p is 8.

4. The method of claim 3, wherein the dose is about 3.0 mg/kg.

5. The method of claim 4, wherein the FRα-positive cancer is selected from the group consisting of ovarian cancer, endometrial cancer, cervical cancer, non-small cell lung cancer (NSCLC), triple-negative breast cancer (TNBC), pancreatic cancer, and colorectal cancer (CRC).

6. The method of claim 5, wherein the ovarian cancer is epithelial ovarian cancer, primary peritoneal cancer, fallopian tube cancer, and high grade endometriod ovarian cancer, low grade serous ovarian cancer (LGSOC), and high grade serous ovarian cancer (HGSOC).

7. The method of claim 5, wherein the ovarian cancer is resistant to prior platinum treatment (PROC).

8. The method of claim 5, wherein the ovarian cancer is platinum sensitive (PSOC).

9. The method of claim 7, wherein the subject was previously treated with mirvetuximab soravtansine-gynx or its biosimilar.

10. The method of claim 7, wherein the subject was previously treated with ≤3 lines of therapy.

11. The method of claim 9, wherein the subject was previously treated with ≤4 lines of therapy.

12. The method of claim 8, wherein the subject was previously treated with ≥2 lines of therapy.

13. The method of claim 7, wherein the subject is aged 65 and over.

14. The method of claim 7, wherein the anti-folate receptor alpha-antibody drug conjugate is administered as a monotherapy.

15. The method of claim 12, wherein the treatment further comprises administering bevacizumab or its biosimilar which is administered after administration of the anti-folate receptor alpha-antibody drug conjugate.

16. The method of claim 15, wherein the dosing regimen for bevacizumab or its biosimilar is administered on Day 1 of every 21-day cycle (Q3W) at a dose of about 15 mg/kg for up to 22 cycles.

17. The method of claim 16, wherein the combination with bevacizumab or its biosimilar is to treat platinum sensitive disease as a second line therapy.

18. The method of claim 14, wherein no pulmonary toxicity is observed in the subject as a result of treatment.

19. The method of claim 14, wherein no neuropathy is observed in the subject as a result of treatment.

20. The method of claim 14, wherein no ocular toxicity is observed in the subject as a result of treatment.

21. The method of claim 1, wherein the FRα-positive cancer is selected from the group consisting of ovarian cancer, endometrial cancer, cervical cancer, non-small cell lung cancer (NSCLC), triple-negative breast cancer (TNBC), pancreatic cancer, and colorectal cancer (CRC).

22. The method of claim 8, wherein the subject is aged 65 and over.

23. The method of claim 16, wherein no pulmonary toxicity is observed in the subject as a result of treatment.

24. The method of claim 16, wherein no neuropathy is observed in the subject as a result of treatment.

25. The method of claim 16, wherein no ocular toxicity is observed in the subject as a result of treatment.
